(19) United States
(12) Patent Application Publication
(10) Pub. No.: US 2023/0055617 A1
(43) Pub. Date: Feb. 23, 2023

(54) Methods and Systems for Engineering Photoplethysmographic-Waveform Features From Biophysical Signals for Use in Characterizing Physiological Systems

(71) Applicant: Analytics for Life Inc., Toronto (CA)

(72) Inventors: Emmanuel Lange, Toronto (CA); Farhad Fathieh, North York (CA)

(21) Appl. No.: 17/891,278

(22) Filed: Aug. 19, 2022

Related U.S. Application Data
(60) Provisional application No. 63/235,971, filed on Aug. 23, 2021.

(51) Int. Cl. A61B 5/00; A61B 5/024; A61B 5/1455; G16H 50/20

(52) U.S. Cl. CPC A61B 5/7282; A61B 5/0022; A61B 5/02416; A61B 5/7264; A61B 5/14551; G16H 50/20; A61B 5/725

(57) ABSTRACT

The exemplified methods and systems facilitate the use, for diagnostics, monitoring, or treatment, of one or more PPG waveform-based features or parameters determined from biophysical signals such as photoplethysmography signals that are acquired non-invasively from surface sensors placed on a patient while the patient is at rest. PPG waveform-based features or parameters may include PPG waveform features or parameters, VPG waveform features or parameters, and/or APG waveform features or parameters. The PPG waveform-based features or parameters can be used in a model or classifier to estimate metrics associated with the physiological state of a patient, including the presence or non-presence of a disease, medical condition, or an indication of either. The estimated metric may be used to assist a physician or other healthcare provider in diagnosing the presence or non-presence and/or severity and/or localization of diseases or conditions or in the treatment of said diseases or conditions.

### RELATED APPLICATION

This US application claims priority to, and the benefit of, U.S. Provisional Pat. Application No. 63/235,971, filed Aug. 23, 2021, entitled “Methods and Systems for Engineering Photoplethysmographic-Waveform Features From Biophysical Signals for Use in Characterizing Physiological Systems,” which is incorporated by reference herein in its entirety.

### FIELD OF THE INVENTIONS

The present disclosure generally relates to methods and systems for engineering features or parameters from biophysical signals for use in diagnostic applications; in particular, the engineering and use of photoplethysmographic waveform-based features for use in characterizing one or more physiological systems and their associated functions, activities, and abnormalities. The features or parameters may also be used for monitoring or tracking, controls of medical equipment, or to guide the treatment of a disease, medical condition, or an indication of either.

### BACKGROUND

There are numerous methods and systems for assisting a healthcare professional in diagnosing disease. Some of these involve the use of invasive or minimally invasive techniques, radiation, exercise or stress, or pharmacological agents, sometimes in combination, with their attendant risks and other disadvantages.

Diastolic heart failure, a major cause of morbidity and mortality, is defined as symptoms of heart failure in a patient with preserved left ventricular function. It is characterized by a stiff left ventricle with decreased compliance and impaired relaxation leading to increased end-diastolic pressure in the left ventricle, which is measured through left heart catheterization. Current clinical standard of care for diagnosing pulmonary hypertension (PH), and for pulmonary arterial hypertension (PAH), in particular, involves a cardiac catheterization of the right side of the heart that directly measures the pressure in the pulmonary arteries. Coronary angiography is the current standard of care used to assess coronary arterial disease (CAD) as determined through the coronary lesions described by a treating physician. Non-invasive imaging systems such as magnetic resonance imaging and computed tomography require specialized facilities to acquire images of blood flow and arterial blockages of a patient that are reviewed by radiologists.

It is desirable to have a system that can assist healthcare professionals in the diagnosis of cardiac disease and various other diseases and conditions without the aforementioned disadvantages.

### SUMMARY

A clinical evaluation system and method are disclosed that facilitate the use of one or more PPG waveform-based features or parameters determined from biophysical signals such as cardiac/biopotential signals and/or photoplethysmography signals that are acquired, in preferred embodiments, non-invasively from surface sensors placed on a patient while the patient is at rest. Photoplethysmographic waveforms may be acquired via a pulse oximeter or similar equipment that illuminates the skin and measures changes in light absorption at two or more distinct wavelengths.

The estimation or determined likelihood of the presence or non-presence of a disease, condition, or indication of either can supplant, augment, or replace other evaluation or measurement modalities for the assessment of a disease or medical condition. In some cases, a determination can take the form of a numerical score and related information.

For a given photoplethysmographic waveform (“PPG waveform”), the first derivative of the PPG waveform may be computed to generate a velocityplethysmogram (VPG) waveform (“VPG waveform”), and a second derivative of the PPG waveform may be computed to generate an accelerationplethysmogram (APG) waveform (“APG waveform”). PPG waveform-based features or parameters may include PPG waveform features or parameters, VPG waveform features or parameters, and/or APG waveform features or parameters. The PPG waveform-based features or parameters can be used in a model or classifier (e.g., a machine-learned classifier) to estimate metrics associated with the physiological state of a patient, including for the presence or non-presence of a disease, medical condition, or an indication of either. The estimated metric may be used to assist a physician or other healthcare provider in diagnosing the presence or non-presence and/or severity and/or localization of diseases or conditions or in the treatment of said diseases or conditions. In some embodiments, the waveforms may be analyzed in the frequency domain.

The exemplary system employs a hemodynamic delineator to identify fiducial points in the PPG waveform(s) and its corresponding VPG and APG waveforms. Waveform (“linear”) features that describe the PPG, VPG, and APG waveform characteristics in the time domain such as amplitude attributes of the fiducial points or duration/geometric attributes among the fiducial points may be determined solely from an acquired PPG signal data set (i.e., without information from other sources).

As used herein, the term “feature” (in the context of machine learning and pattern recognition and as used herein) generally refers to an individual measurable property or characteristic of a phenomenon being observed. A feature is defined by analysis and may be determined in groups in combination with other features from a common model or analytical framework.

As used herein, “metric” refers to an estimation or likelihood of the presence, non-presence, severity, and/or localization (where applicable) of one or more diseases, conditions, or indication(s) of either, in a physiological system or systems. Notably, the exemplified methods and systems can be used in certain embodiments described herein to acquire biophysical signals and/or to otherwise collect data from a patient and to evaluate those signals and/or data in signal processing and classifier operations to evaluate for a disease, condition, or indicator of one that can supplant, augment, or replace other evaluation modalities via one or more metrics. In some cases, a metric can take the form of a numerical score and related information.

In the context of cardiovascular and respiratory systems, examples of diseases and conditions to which such metrics can relate include, for example: (i) heart failure (e.g., left-side or right-side heart failure; heart failure with preserved ejection fraction (HFpEF)), (ii) coronary artery disease (CAD), (iii) various forms of pulmonary hypertension (PH) including without limitation pulmonary arterial hypertension (PAH), (iv) abnormal left ventricular ejection fraction (LVEF), and various other diseases or conditions. An example indicator of certain forms of heart failure is the presence or non-presence of elevated or abnormal left-ventricular end-diastolic pressure (LVEDP). An example indicator of certain forms of pulmonary hypertension is the presence or non-presence of elevated or abnormal mean pulmonary arterial pressure (mPAP).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate embodiments and, together with the description, serve to explain the principles of the methods and systems.

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIG. 1 is a schematic diagram of example modules, or components, configured to non-invasively compute PPG waveform-based features or parameters to generate one or more metrics associated with the physiological state of a patient in accordance with an illustrative embodiment.

FIG. 2 shows an example biophysical signal capture system or component and its use in non-invasively collecting biophysical signals of a patient in a clinical setting in accordance with an illustrative embodiment.

FIGS. 3A-3B each shows an example method to use PPG waveform-based features/parameters or their intermediate data in a practical application for diagnostics, treatment, monitoring, or tracking in accordance with an illustrative embodiment.

FIG. 4 illustrates an example PPG amplitude feature computation module configured to determine amplitude associated attributes of an acquired PPG signal(s) and its corresponding VPG and APG signals in accordance with an illustrative embodiment.

FIG. 5 illustrates an example PPG duration feature computation module configured to determine duration associated attributes of an acquired PPG signal and its corresponding VPG and APG signals in accordance with an illustrative embodiment.

FIG. 6 illustrates an example PPG geometric feature computation module configured to determine geometric attributes of an acquired PPG signal and its corresponding VPG and APG signals in accordance with an illustrative embodiment.

FIG. 7 illustrates an example PPG SpO₂ feature computation module configured to determine SpO₂ associated attributes of an acquired PPG signal in accordance with an illustrative embodiment.

FIG. 8A shows an example method of the feature computation module of FIG. 4 or that of FIG. 5 in accordance with an illustrative embodiment.

FIG. 8B shows an example method of the feature computation module of FIG. 6 in accordance with an illustrative embodiment.

FIGS. 9A-9D and 10A-10C each shows various aspects of the operation of a hemodynamic delineator to perform the method of FIGS. 8A or 8B, among others, in accordance with an illustrative embodiment.

FIG. 11 shows various aspects of determining PPG duration features or parameters in accordance with an illustrative embodiment.

FIG. 12 shows various aspects of determining PPG geometric features of parameters in accordance with an illustrative embodiment.

FIG. 13 shows an aspect of determining PPG SpO₂ features or parameters in accordance with an illustrative embodiment.

FIG. 14A shows a schematic diagram of an example clinical and diagnostic evaluation system configured to use PPG waveform-based features or parameters among other computed features to generate one or more metrics associated with the physiological state of a patient in accordance with an illustrative embodiment.

FIG. 14B shows a schematic diagram of the operation of the example clinical evaluation system of FIG. 14A in accordance with an illustrative embodiment.

### DETAILED DESCRIPTION

Each and every feature described herein, and each and every combination of two or more of such features, is included within the scope of the present invention provided that the features included in such a combination are not mutually inconsistent.

While the present disclosure is directed to the practical assessment of biophysical signals, e.g., raw or pre-processed photoplethysmographic signals, biopotential/cardiac signals, etc., in the diagnosis, tracking, and treatment of cardiac-related pathologies and conditions, such assessment can be applied to the diagnosis, tracking, and treatment (including without limitation surgical, minimally invasive, lifestyle, nutritional, and/or pharmacologic treatment, etc.) of any pathologies or conditions in which a biophysical signal is involved in any relevant system of a living body. The assessment may be used in the controls of medical equipment or wearable devices or in monitoring applications (e.g., to report the PPG waveform-based features, parameters, or an intermediate output discussed herein)

The terms “subject” and “patient” as used herein are generally used interchangeably to refer to those who had undergone analysis performed by the exemplary systems and methods.

The term “cardiac signal” as used herein refers to one or more signals directly or indirectly associated with the structure, function, and/or activity of the cardiovascular system -including aspects of that signal’s electrical/electrochemical conduction - that, e.g., cause contraction of the myocardium. A cardiac signal may include, in some embodiments, biopotential signals or electrocardiographic signals, e.g., those acquired via an electrocardiogram (ECG), the cardiac and photoplethysmographic waveform or signal capture or recording instrument later described herein, or other modalities.

The term “biophysical signal” as used herein includes but is not limited to one or more cardiac signal(s), neurological signal(s), ballistocardiographic signal(s), and/or photoplethysmographic signal(s), but it also encompasses more broadly any physiological signal from which information may be obtained. Not intending to be limited by example, one may classify biophysical signals into types or categories that can include, for example, electrical (e.g., certain cardiac and neurological system-related signals that can be observed, identified, and/or quantified by techniques such as the measurement of voltage/potential (e.g., biopotential), impedance, resistivity, conductivity, current, etc. in various domains such as time and/or frequency), magnetic, electromagnetic, optical (e.g., signals that can be observed, identified and/or quantified by techniques such as reflectance, interferometry, spectroscopy, absorbance, transmissivity, visual observation, photoplethysmography, and the like), acoustic, chemical, mechanical (e.g., signals related to fluid flow, pressure, motion, vibration, displacement, strain), thermal, and electrochemical (e.g., signals that can be correlated to the presence of certain analytes, such as glucose). Biophysical signals may in some cases be described in the context of a physiological system (e.g., respiratory, circulatory (cardiovascular, pulmonary), nervous, lymphatic, endocrine, digestive, excretory, muscular, skeletal, renal/urinary/excretory, immune, integumentary/exocrine and reproductive systems), one or more organ system(s) (e.g., signals that may be unique to the heart and lungs as they work together), or in the context of tissue (e.g., muscle, fat, nerves, connective tissue, bone), cells, organelles, molecules (e.g., water, proteins, fats, carbohydrates, gases, free radicals, inorganic ions, minerals, acids, and other compounds, elements, and their subatomic components. Unless stated otherwise, the term “biophysical signal acquisition” generally refers to any passive or active means of acquiring a biophysical signal from a physiological system, such as a mammalian or non-mammalian organism. Passive and active biophysical signal acquisition generally refers to the observation of natural or induced electrical, magnetic, optical, and/or acoustics emittance of the body tissue. Non-limiting examples of passive and active biophysical signal acquisition means include, e.g., voltage/potential, current, magnetic, optical, acoustic, and other non-active ways of observing the natural emittance of the body tissue, and in some instances, inducing such emittance. Non-limiting examples of passive and active biophysical signal acquisition means include, e.g., ultrasound, radio waves, microwaves, infrared and/or visible light (e.g., for use in pulse oximetry or photoplethysmography), visible light, ultraviolet light, and other ways of actively interrogating the body tissue that does not involve ionizing energy or radiation (e.g., X-ray). An active biophysical signal acquisition may involve excitation-emission spectroscopy (including, for example, excitation-emission fluorescence). The active biophysical signal acquisition may also involve transmitting ionizing energy or radiation (e.g., X-ray) (also referred to as “ionizing biophysical signal”) to the body tissue. Passive and active biophysical signal acquisition means can be performed in conjunction with invasive procedures (e.g., via surgery or invasive radiologic intervention protocols) or non-invasively (e.g., via imaging, ablation, heart contraction regulation (e.g., via pacemakers), catheterization, etc.).

The term “photoplethysmographic signal” as used herein refers to one or more signals or waveforms acquired from optical sensors that correspond to measured changes in light absorption by oxygenated and deoxygenated hemoglobin, such as light having wavelengths in the red and infrared spectra. Photoplethysmographic signal(s), in some embodiments, include a raw signal(s) acquired via a pulse oximeter or a photoplethysmogram (PPG). In some embodiments, photoplethysmographic signal(s) are acquired from off-the-shelf, custom, and/or dedicated equipment or circuitries that are configured to acquire such signal waveforms for the purpose of monitoring health and/or diagnosing disease or abnormal conditions. The photoplethysmographic signal(s) typically include a red photoplethysmographic signal (e.g., an electromagnetic signal in the visible light spectrum most dominantly having a wavelength of approximately 625 to 740 nanometers) and an infrared photoplethysmographic signal (e.g., an electromagnetic signal extending from the nominal red edge of the visible spectrum up to about 1 mm), though other spectra such as near-infrared, blue and green may be used in different combinations, depending on the type and/or mode of PPG being employed.

The term “ballistocardiographic signal,” as used herein, refers to a signal or group of signals that generally reflect the flow of blood through the entire body that may be observed through vibration, acoustic, movement, or orientation. In some embodiments, ballistocardiographic signals are acquired by wearable devices, such as vibration, acoustic, movement, or orientation-based seismocardiogram (SCG) sensors, which can measure the body’s vibrations or orientation as recorded by sensors mounted close to the heart. Seismocardiogram sensors are generally used to acquire “seismocardiogram,” which is used interchangeably with the term “ballistocardiogram” herein. In other embodiments, ballistocardiographic signals may be acquired by external equipment, e.g., bed or surface-based equipment that measures phenomena such as a change in body weight as blood moves back and forth in the longitudinal direction between the head and feet. In such embodiments, the volume of blood in each location may change dynamically and be reflected in the weight measured at each location on the bed as well as the rate of change of that weight.

In addition, the methods and systems described in the various embodiments herein are not so limited and may be utilized in any context of another physiological system or systems, organs, tissue, cells, etc., of a living body. By way of example only, two biophysical signal types that may be useful in the cardiovascular context include cardiac/biopotential signals that may be acquired via conventional electrocardiogram (ECG/EKG) equipment, bipolar wide-band biopotential (cardiac) signals that may be acquired from other equipment such as those described herein, and signals that may be acquired by various plethysmographic techniques, such as, e.g., photoplethysmography. In another example, the two biophysical signal types can be further augmented by ballistocardiographic techniques.

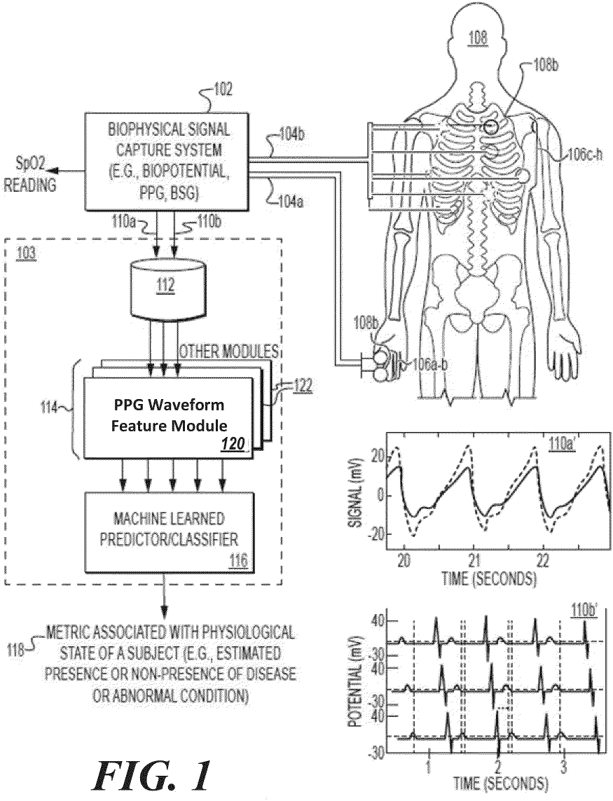

FIG. 1 is a schematic diagram of example modules, or components, configured to non-invasively compute PPG waveform-based features or parameters to generate, via a classifier (e.g., machine-learned classifier), one or more metrics associated with the physiological state of a patient in accordance with an illustrative embodiment. The modules or components may be used in a production application or the development of the PPG waveform-based features and other classes of features.

The example analysis and classifiers described herein may be used to assist a healthcare provider in the diagnosis and/or treatment of cardiac- and cardiopulmonary-related pathologies and medical conditions, or an indicator of one. Examples include significant coronary artery disease (CAD), one or more forms of heart failure such as, e.g., heart failure with preserved ejection fraction (HFpEF), congestive heart failure, various forms of arrhythmia, valve failure, various forms of pulmonary hypertension, among various other disease and conditions disclosed herein.

In addition, there exist possible indicators of a disease or condition, such as an elevated or abnormal left ventricular end-diastolic pressure (LVEDP) value as it relates to some forms of heart failure, abnormal left ventricular ejection fraction (LVEF) values as they relate to some forms of heart failure or an elevated mean pulmonary arterial pressure (mPAP) value as it relates to pulmonary hypertension and/or pulmonary arterial hypertension. Indicators of the likelihood that such indicators are abnormal/elevated or normal, such as those provided by the example analysis and classifiers described herein, can help a healthcare provider assess or diagnose that the patient has or does not have a given disease or condition. In addition to these metrics associated with a disease state of condition, other measurements and factors may be employed by a healthcare professional in making a diagnosis, such as the results of a physical examination and/or other tests, the patient’s medical history, current medications, etc. The determination of the presence or non-presence of a disease state or medical condition can include the indication (or a metric of measure that is used in the diagnosis) for such disease.

In FIG. 1, the components include at least one non-invasive biophysical signal recorder or capture system 102 and an assessment system 103 that is located, for example, in a cloud or remote infrastructure or in a local system. Biophysical signal capture system 102 (also referred to as a biophysical signal recorder system), in this embodiment, is configured to, e.g., acquire, process, store and transmit synchronously acquired patient’s electrical and hemodynamic signals as one or more types of biophysical signals 104. In the example of FIG. 1, the biophysical signal capture system 102 is configured to synchronously capture two types of biophysical signals shown as first biophysical signals 104a (e.g., synchronously acquired to other first biophysical signals) and second biophysical signals 104b (e.g., synchronously acquired to the other biophysical signals) acquired from measurement probes 106 (e.g., shown as probes 106a and 106b, e.g., comprising hemodynamic sensors for hemodynamic signals 104a, and probes 106c-106h comprising leads for electrical/cardiac signals 104b). The probes 106a-h are placed on, e.g., by being adhered to or placed next to, a surface tissue of a patient 108 (shown at patient locations 108a and 108b). The patient is preferably a human patient, but it can be any mammalian patient. The acquired raw biophysical signals (e.g., 106a and 106b) together form a biophysical-signal data set 110 (shown in FIG. 1 as a first biophysical-signal data set 110a and a second biophysical-signal data set 110b, respectively) that may be stored, e.g., as a single file, preferably, that is identifiable by a recording/signal captured number and/or by a patient’s name and medical record number.

In the FIG. 1 embodiment, the first biophysical-signal data set 110a comprises a set of raw photoplethysmographic, or hemodynamic, signal(s) associated with measured changes in light absorption of oxygenated and/or deoxygenated hemoglobin from the patient at location 108a, and the second biophysical-signal data set 110b comprises a set of raw cardiac or biopotential signal(s) associated with electrical signals of the heart. Though in FIG. 1, raw photoplethysmographic or hemodynamic signal(s) are shown being acquired at a patient’s finger, the signals may be alternatively acquired at the patient’s toe, wrist, forehead, earlobe, neck, etc. Similarly, although the cardiac or biopotential signal(s) are shown to be acquired via three sets of orthogonal leads, other lead configurations may be used (e.g., 11 lead configuration, 12 lead configuration, etc.).

Plots 110a' and 110b' show examples of the first biophysical-signal data set 110a and the second biophysical-signal data set 110a, respectively. Specifically, Plot 110a' shows an example of an acquired photoplethysmographic or hemodynamic signal. In Plot 110a', the photoplethysmographic signal is a time series signal having a signal voltage potential as a function of time as acquired from two light sources (e.g., infrared and red-light source). Plot 110b' shows an example cardiac signal comprising a 3-channel potential time series plot. In some embodiments, the biophysical signal capture system 102 preferably acquires biophysical signals via non-invasive means or component(s). In alternative embodiments, invasive or minimally-invasively means or component(s) may be used to supplement or as substitutes for the non-invasive means (e.g., implanted pressure sensors, chemical sensors, accelerometers, and the like). In still further alternative embodiments, non-invasive and non-contact probes or sensors capable of collecting biophysical signals may be used to supplement or as substitutes for the non-invasive and/or invasive/minimally invasive means, in any combination (e.g., passive thermometers, scanners, cameras, x-ray, magnetic, or other means of non-contact or contact energy data collection system as discussed herein). Subsequent to signal acquisitions and recording, the biophysical signal capture system 102 then provides, e.g., sending over a wireless or wired communication system and/or a network, the acquired biophysical-signal data set 110 (or a data set derived or processed therefrom, e.g., filtered or pre-processed data) to a data repository 112 (e.g., a cloud-based storage area network) of the assessment system 103. In some embodiments, the acquired biophysical-signal data set 110 is sent directly to the assessment system 103 for analysis or is uploaded to a data repository 112 through a secure clinician’s portal.

Biophysical signal capture system 102 is configured with circuitries and computing hardware, software, firmware, middleware, etc., in some embodiments, to acquire, store, transmit, and optionally process both the captured biophysical signals to generate the biophysical-signal data set 110. An example biophysical signal capture system 102 and the acquired biophysical-signal set data 110 are described in U.S. Pat. No. 10,542,898, entitled “Method and Apparatus for Wide-Band Phase Gradient Signal Acquisition,” or U.S. Pat. Publication No. 2018/0249960, entitled “Method and Apparatus for Wide-Band Phase Gradient Signal Acquisition,” each of which is hereby incorporated by reference herein in its entirety.

In some embodiments, biophysical signal capture system 102 includes two or more signal acquisition components, including a first signal acquisition component (not shown) to acquire the first biophysical signals (e.g., photoplethysmographic signals) and includes a second signal acquisition component (not shown) to acquire the second biophysical signals (e.g., cardiac signals). In some embodiments, the electrical signals are acquired at a multi-kilohertz rate for a few minutes, e.g., between 1 kHz and 10 kHz. In other embodiments, the electrical signals are acquired between 10 kHz and 100 kHz. The hemodynamic signals may be acquired, e.g., between 100 Hz and 1 kHz.

Biophysical signal capture system 102 may include one or more other signal acquisition components (e.g., sensors such as mechano-acoustic, ballistographic, ballistocardiographic, etc.) for acquiring signals. In other embodiments of the signal capture system 102, a signal acquisition component comprises conventional electrocardiogram (ECG/EKG) equipment (e.g., Holter device, 12 lead ECG, etc.).

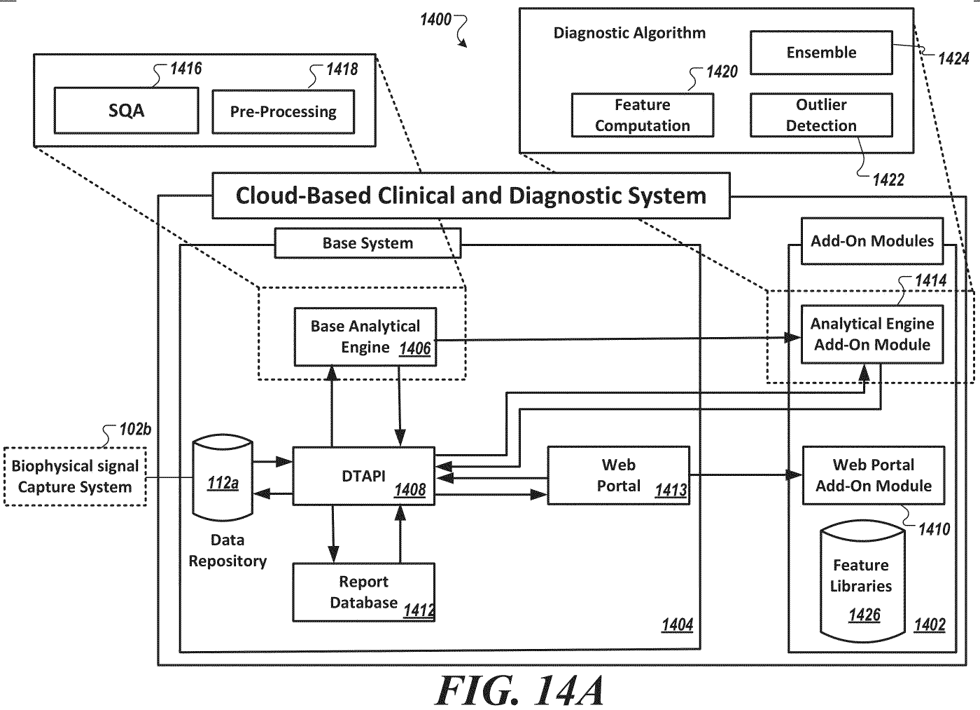

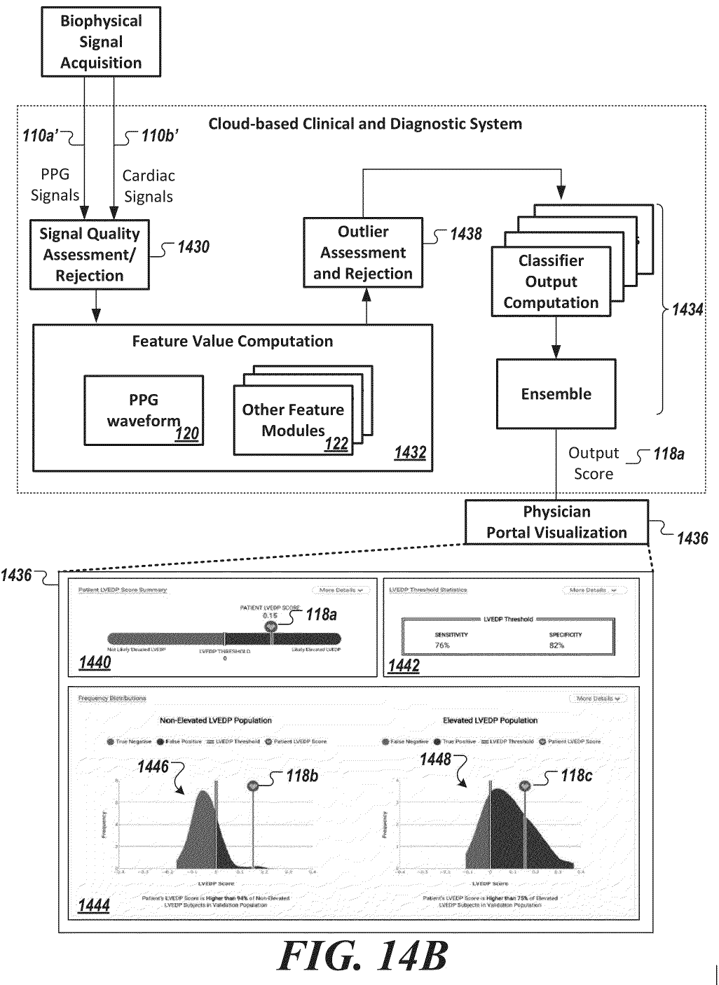

Assessment system 103 comprises, in some embodiments, the data repository 112 and an analytical engine or analyzer (not shown - see FIGS. 14A and 14B). Assessment system 103 may include feature modules 114 and a classifier module 116 (e.g., an ML classifier module). In FIG. 1, Assessment system 103 is configured to retrieve the acquired biophysical signal data set 110, e.g., from the data repository 112, and use it in the feature modules 114, which is shown in FIG. 1 to include a PPG-waveform feature module 120 and other modules 122 (later described herein). The features modules 114 compute values of features or parameters, including those of PPG waveform-based features to provide to the classifier module 116, which computes an output 118, e.g., an output score, of the metrics associated with the physiological state of a patient (e.g., an indication of the presence or non-presence of a disease state, medical condition, or an indication of either). Output 118 is subsequently presented, in some embodiments, at a healthcare physician portal (not shown - see FIGS. 14A and 14B) to be used by healthcare professionals for the diagnosis and treatment of pathology or a medical condition. In some embodiments, a portal may be configured (e.g., tailored) for access by, e.g., patients, caregivers, researchers, etc., with output 118 configured for the portal’s intended audience. Other data and information may also be a part of output 118 (e.g., the acquired biophysical signals or other patient’s information and medical history).

Classifier module 116 (e.g., ML classifier module) may include transfer functions, look-up tables, models, or operators developed based on algorithms such as but not limited to decision trees, random forests, neural networks, linear models, Gaussian processes, nearest neighbor, SVMs, Naive Bayes, etc. In some embodiments, classifier module 116 may include models that are developed based on ML techniques described in U.S. Provisional Pat. Application no. 63/235,960, filed Aug. 23, 2021, entitled “Method and System to Non-Invasively Assess Elevated Left Ventricular End-Diastolic Pressure”; U.S. Pat. Publication No. 20190026430, entitled “Discovering Novel Features to Use in Machine Learning Techniques, such as Machine Learning Techniques for Diagnosing Medical Conditions”; or U.S. Pat. Publication No. 20190026431, entitled “Discovering Genomes to Use in Machine Learning Techniques,” each of which is hereby incorporated by reference herein in its entirety.

### Example Biophysical Signal Acquisition

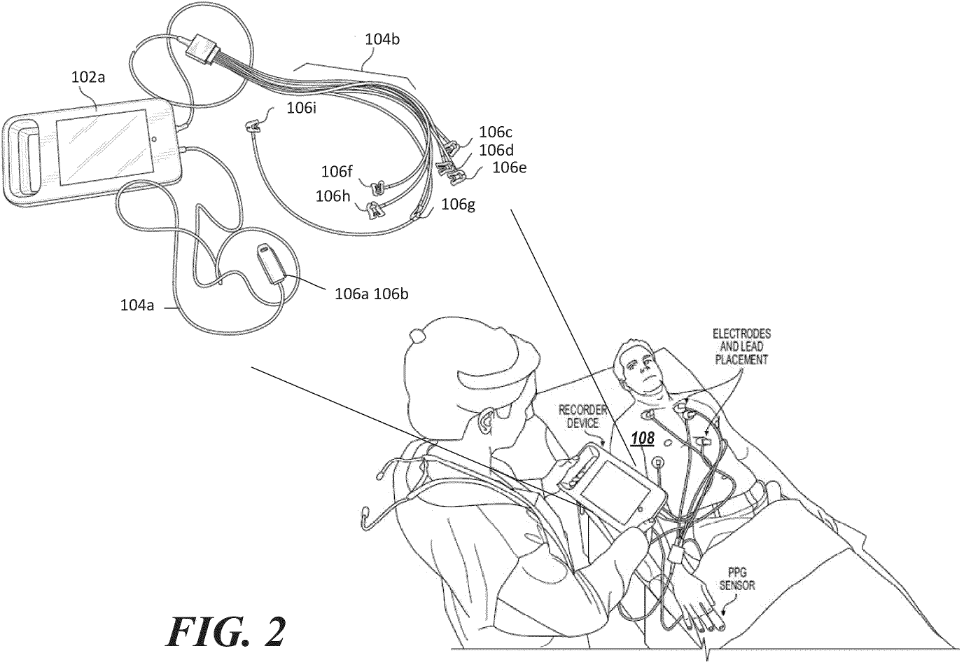

FIG. 2 shows a biophysical signal capture system 102 (shown as 102a) and its use in non-invasively collecting biophysical signals of a patient in a clinical setting in accordance with an illustrative embodiment. In FIG. 2, the biophysical signal capture system 102a is configured to capture two types of biophysical signals from the patient 108 while the patient is at rest. The biophysical signal capture system 102a synchronously acquires the patient’s (i) electrical signals (e.g., cardiac signals corresponding to the second biophysical-signal data set 110b) from the torso using orthogonally placed sensors (106c-106h; 106i is a 7^{th} common-mode reference lead) and (ii) hemodynamic signals (e.g., PPG signals corresponding to the first biophysical-signal data set 110a) from the finger using a photoplethysmographic sensor (e.g., collecting signals 106a, 106b).

As shown in FIG. 2, the electrical and hemodynamic signals (e.g., 104a, 104b) are passively collected via commercially available sensors applied to the patient’s skin. The signals may be acquired beneficially without patient exposure to ionizing radiation or radiological contrast agents and without patient exercise or the use of pharmacologic stressors. The biophysical signal capture system 102a can be used in any setting conducive for a healthcare professional, such as a technician or nurse, to acquire the requisite data and where a cellular signal or Wi-Fi connection can be established.

The electrical signals (e.g., corresponding to the second biophysical signal data set 110b) are collected using three orthogonally paired surface electrodes arranged across the patient’s chest and back along with a reference lead. The electrical signals are acquired, in some embodiments, using a low-pass anti-aliasing filter (e.g., ~ 2 kHz) at a multi-kilohertz rate (e.g., 8 thousand samples per second for each of the six channels) for a few minutes (e.g., 215 seconds). In alternative embodiments, the biophysical signals may be continuously/intermittently acquired for monitoring, and portions of the acquired signals are used for analysis. The hemodynamic signals (e.g., corresponding to the first biophysical signal data set 110a) are collected using a photoplethysmographic sensor placed on a finger. The photo-absorption of red light (e.g., any wavelengths between 600-750 nm) and infrared light (e.g., any wavelengths between 850-950 nm) are recorded, in some embodiments, at a rate of 500 samples per second over the same period. The biophysical signal capture system 102a may include a common mode drive that reduces common-mode environmental noise in the signal. The photoplethysmographic and cardiac signals were simultaneously acquired for each patient. Jitter (inter-modality jitter) in the data may be less than about 10 microseconds (µs). Jitter among the cardiac signal channels may be less than 10 microseconds, e.g., around ten femtoseconds (fs).

A signal data package containing the patient metadata and signal data may be compiled at the completion of the signal acquisition procedure. This data package may be encrypted before the biophysical signal capture system 102a transfers the package to the data repository 112. In some embodiments, the data package is transferred to the assessment system (e.g., 103). The transfer is initiated, in some embodiments, following the completion of the signal acquisition procedure without any user intervention. The data repository 112 is hosted, in some embodiments, on a cloud storage service that can provide secure, redundant, cloud-based storage for the patient’s data packages, e.g., Amazon Simple Storage Service (i.e., “Amazon S3”). The biophysical signal capture system 102a also provides an interface for the practitioner to receive notification of an improper signal acquisition to alert the practitioner to immediately acquire additional data from the patient.

### Example Method of Operation

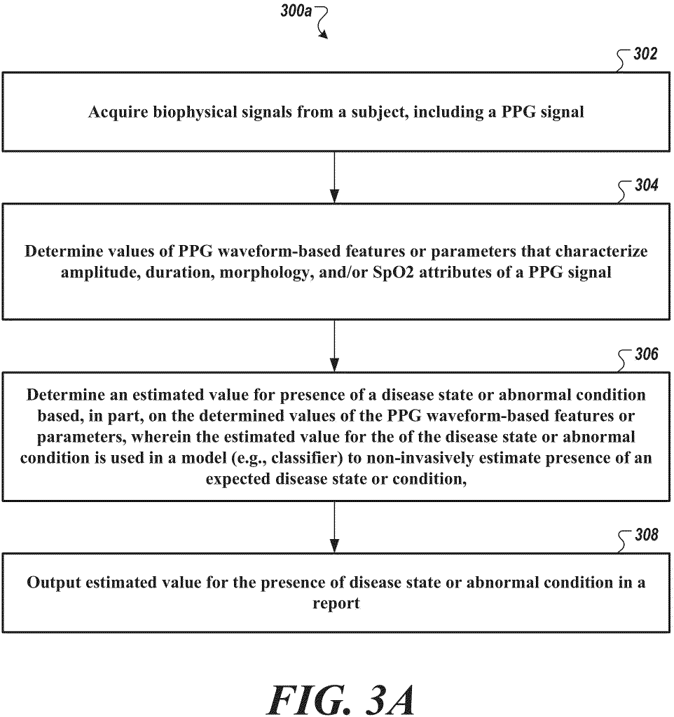

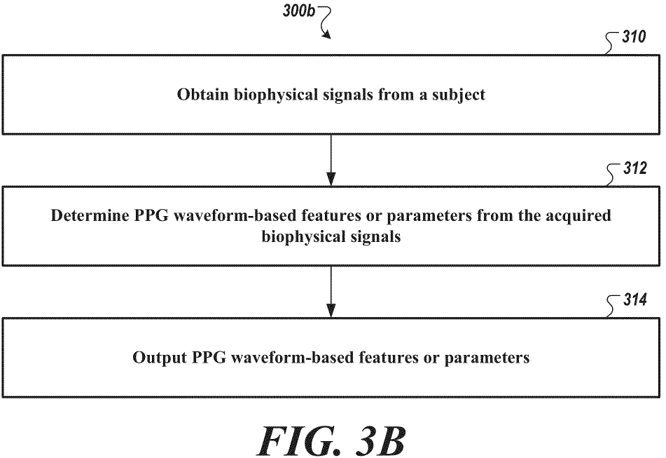

FIGS. 3A-3B each shows an example method to use PPG waveform-based features/parameters or their intermediate outputs in a practical application for diagnostics, treatment, monitoring, or tracking.

Estimation of Presence of Disease State or Indicating Condition. FIG. 3A shows a method 300a that employs PPG waveform-based parameters or features to determine estimators of the presence of a disease state, medical condition, or indication of either, e.g., to aid in the diagnosis, tracking, or treatment. Method 300a includes the step of acquiring (302) biophysical signals from a patient (e.g., cardiac signals, photoplethysmographic signals, ballistocardiographic signals), e.g., as described in relation to FIGS. 1 and 2 and other examples as described herein. In some embodiments, the acquired biophysical signals are transmitted for remote storage and analysis. In other embodiments, the acquired biophysical signals are stored and analyzed locally.

As stated above, one example in the cardiac context is the estimation of the presence of abnormal left-ventricular end-diastolic pressure (LVEDP) or mean pulmonary artery pressure (mPAP), significant coronary artery disease (CAD), abnormal left ventricular ejection fraction (LVEF), and one or more forms of pulmonary hypertension (PH), such as pulmonary arterial hypertension (PAH). Other pathologies or indicating conditions that may be estimated include, e.g., one or more forms of heart failure such as, e.g., heart failure with preserved ejection fraction (HFpEF), arrhythmia, congestive heart failure, valve failure, among various other diseases and medical conditions disclosed herein.

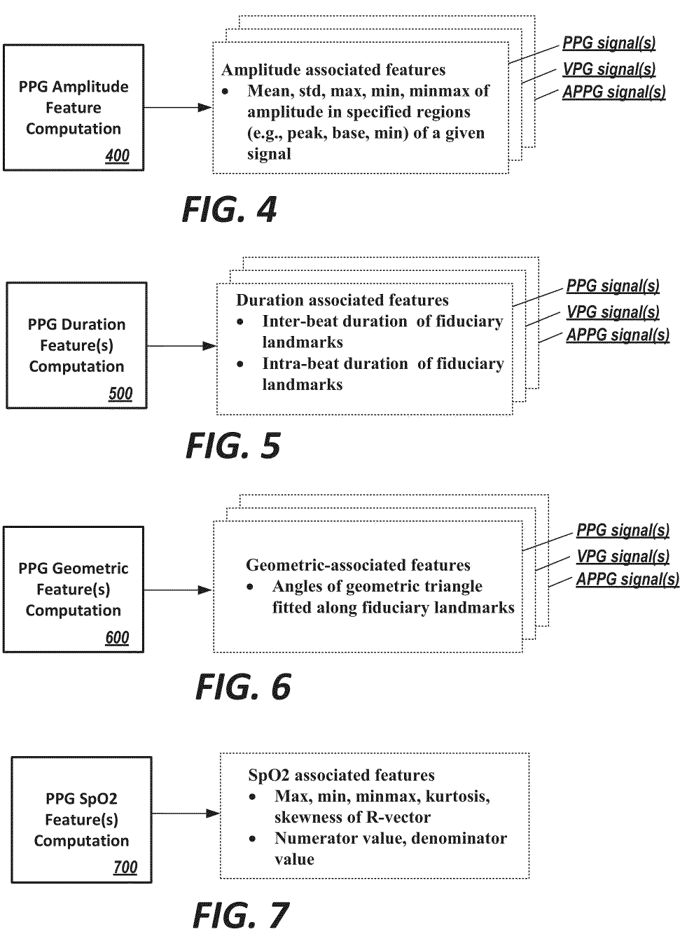

Method 300a further includes the step of retrieving (304) the data set and determining values of PPG waveform-based features or parameters that characterize waveform attributes such as amplitude, duration, and geometry of fiducial points in a PPG, VPG, and/or APG waveform. Example operations to determine the values of PPG waveform-based features or parameters are provided in relation to FIGS. 4-12 later discussed herein. Method 300a further includes the step of determining (306) an estimated value for a presence of a disease state, medical condition, or an indication of either based on an application of the determined PPG waveform-based features to an estimation model (e.g., ML models). An example implementation is provided in relation to FIGS. 14A and 14B.

Method 300a further includes the step of outputting (308) estimated value(s) for the presence of disease state or abnormal condition in a report (e.g., to be used diagnosis or treatment of the disease state, medical condition, or indication of either), e.g., as described in relation to FIGS. 1, 14A, and 14B and other examples described herein.

Diagnostics or Condition Monitoring or Tracking using PPG Waveform-based Features or Parameters. FIG. 3B shows a method 300b that employs PPG waveform-based features or parameters or features for the monitoring or controls of medical equipment or health monitoring device. Method 300b includes the step of obtaining (302) biophysical signals from a patient (e.g., cardiac signals, photoplethysmographic signals, ballistocardiographic signals, etc.). The operation may be performed continuously or intermittently, e.g., to provide output for a report or as controls for the medical equipment or the health monitoring device.

Method 300b further includes determining (310) PPG waveform-based features or parameters from the acquired biophysical data set, e.g., as described in relation to FIGS. 4-12. The determination based may be based on an analysis of the continuously acquired signals over a moving window.

Method 300b further includes outputting (312) PPG waveform-based features or parameters (e.g., in a report for use in diagnostics or as signals for controls). As discussed herein, the PPG waveform-based features or parameters can provide a characterization of attributes such as amplitude, duration, and geometry of fiducial points in a PPG, VPG, or APG waveform. For monitoring and tracking, the output may be via a wearable device, a handheld device, or medical diagnostic equipment (e.g., pulse oximeter system, wearable health monitoring systems). In some embodiments, the output may be via a point of care monitoring, such as a mobile cart or cart trolley. In some embodiments, the outputs may be used in resuscitation systems, cardiac or pulmonary stress test equipment, pacemakers, or other equipment or application.

### PPG Waveform Features or Parameters

FIGS. 4, 5, 6, and 7 each shows an example PPG waveform analysis feature computation module, for a total of four example modules configured to determine values of PPG waveform features or parameters of biophysical signals in accordance with an illustrative embodiment. The PPG amplitude analysis feature computation module 400 can calculate statistical attributes (e.g., mean, standard deviation, maximum, minimum, and/or min-max) of amplitude values of fiduciary points (e.g., peak, base, minimum) across multiple cycles of a PPG, VPG, and APG waveform. The PPG duration analysis feature computation module 500 can calculate duration values between fiduciary points (e.g., peak, base, minimum) of the PPG, VPG, and APG waveforms, to which statistical attributes may also be determined. The PPG geometric analysis feature computation module 600 can calculate topologic values (e.g., angle) of a polygon defined among fiduciary points (e.g., peak, base, minimum) of the PPG, VPG, and APG signals. The PPG SpO₂ analysis feature computation module 700 can calculate values associated with oxygen saturation from the PPG waveform.

### Example #1 - PPG, VPG, APG Amplitude Features

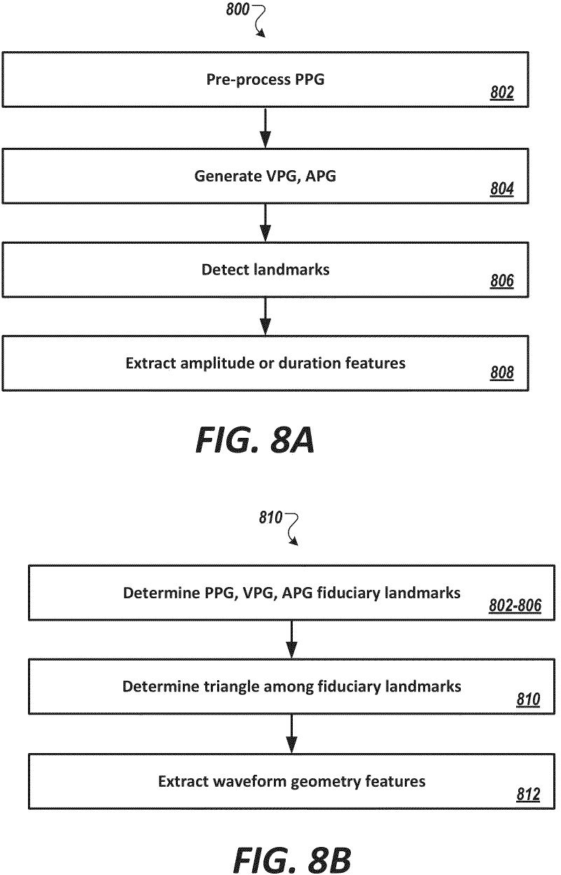

FIG. 4 illustrates, as the first of four example feature or parameter categories, an example PPG amplitude analysis feature computation module 400 configured to determine statistical attributes (e.g., mean, standard deviation, maximum, minimum, and/or min-max) of amplitude values of fiduciary points (e.g., peak, base, minimum) across multiple cycles of a PPG, VPG, and APG waveform. FIG. 8A, discussed below, shows an example method of Module 400.

Table 1 shows an example set of 3 types of extractable waveform amplitude features based on their fiduciary points and their corresponding description to provide up to 45 features or parameters.

Table 1

| | |
|---|---|
| Fiduciary Points for Features | Features |
| pulseBase | Statistical attributes (e.g., mean, standard deviation, maximum, minimum, max-min) of an amplitude distribution of pulse base fiduciary points in a PPG, VPG, or APG signal. |
| systPeak | Statistical attributes (e.g., mean, standard deviation, maximum, minimum, max-min) of an amplitude distribution of systolic peak fiduciary points in a PPG, VPG, or APG signal. |
| diasPeak | Statistical attributes (e.g., mean, standard deviation, maximum, minimum, minimum to maximum ratio ) of an amplitude distribution of diastolic peak fiduciary points in a PPG, VPG, or APG signal. |

Tables 2A and 2B show a summarized set of 30 PPG waveform-based features (“Parameters”) for two PPG waveforms (from 2 PPG measurement channels) and an additional 60 features for the corresponding set of VPG and APG waveforms. In Tables 2A and 2B, 17 feature types (see “*” in Tables 2A and 2B) have been observed to have significant utility in the assessment of the presence or non-presence of at least one cardiac disease or condition -specifically, the determination of presence or non-presence of elevated LVEDP. It has also been observed through experimentation that the 25 feature types (see “**” in Tables 2A and 2B) have significant utility in the assessment of the presence or non-presence of coronary artery disease. The list of the specific features determined to have significant utility in the assessment of the presence or non-presence of abnormal or elevated LVEDP and the presence or non-presence of significant CAD is provided in Table 9A and Table 10A, respectively.

Table 2A

| | |
|---|---|
| Feature Name | Signals to which features are extracted |
| mean pulseBase*'** | PPG waveform of PPG channel #1 (upPPG) |
| std pulseBase** | PPG waveform of PPG channel #2 (lowerPPG) |
| max pulseBase*'** | |
| min pulseBase*'** | |
| minMax pulseBase** | |
| mean systPeak*'** | |
| std systPeak*'** | |
| Max systPeak*'** | |
| min systPeak*'** | |
| minMax systPeak | |
| mean diasPeak** | |
| std diasPeak** | |
| max diasPeak** | |
| min diasPeak** | |
| minMax diasPeak | |

Table 2B

| | |
|---|---|
| Feature Name | Signals to which features are extracted |
| mean peak*'** | VPG waveform of PPG channel #1 (upVPG) |
| std peak** | VPG waveform of PPG channel #2 (lowerVPG) |
| max peak*'** | APG waveform of PPG channel #1 (upAPG) |
| min peak*'** | APG waveform of PPG channel #2 (lowerAPG) |
| minMax peak** | |
| mean min*'** | |
| std min | |
| max min*'** | |
| min min*'** | |
| minMax min** | |
| mean base*'** | |
| std base | |
| max base*'** | |
| min base*'** | |
| minMax base*'** | |

FIG. 8A shows a method 800 to generate PPG waveform-based features or parameters, e.g., as performed by the PPG amplitude feature computation module 400 of FIG. 4, in accordance with an illustrative embodiment, which can be used wholly, or partially, to generate PPG-waveform based features or parameters and its outputs to be used in machine-learned classifier to determine a metric associated with a physiological system of a subject under study. To determine the features of Tables 2A and 2B, Module 400 is configured, in some embodiments, to (i) pre-process (802) the acquired PPG signal, (ii) generate (804) a VPG and APG waveforms from the PPG signal, (iii) determine (806) fiduciary landmarks in the PPG, VPG, and APG waveforms, and (iv) extract (808) features or parameters (e.g., amplitude or duration features or parameters) using the fiduciary landmarks.

To perform the pre-processing operation (802), Module 400 may perform sub-signaling, down-sampling, high-frequency noise and powerline interference removal, and baseline removal operations. To remove the baseline, Module 40 may employ a high-pass filter (e.g., a high-pass filter with a cut-off frequency of 0.3 Hz). To remove high-frequency noise and powerline interference, Module 400 may employ a low-pass filter (e.g., a low-pass digital filter, e.g., with passband frequency of 30 Hz, a stopband frequency of 35 Hz, a passband ripper of 1 dB, and a stopband attenuation of 80 dB. Module 400 may also perform a smoothing operation (e.g., having a centered moving average of 6 points).

To generate the VPG and APG waveforms (804), Module 400 may (i) invert the PPG to generate an absorptive PPG signal, (ii) perform a first derivative operation of the PPG waveform to generate the VPG waveform, and (iii) perform a second derivative of the PPG waveform (or a first derivative of the VPG waveform) to generate the APG waveform. e.g., the adsorptive PPG signal). Any number of PPG, VPG, and/or AGP waveforms may be analyzed.

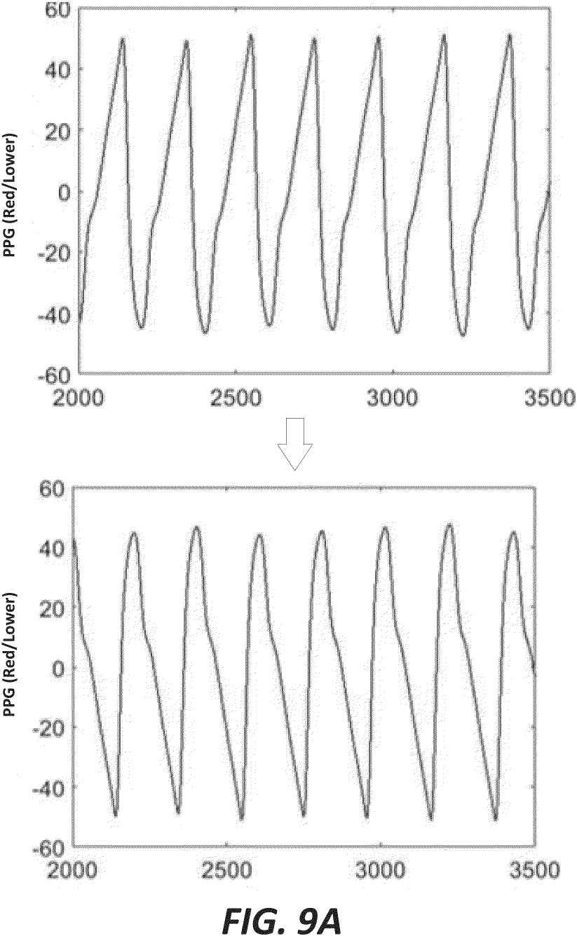

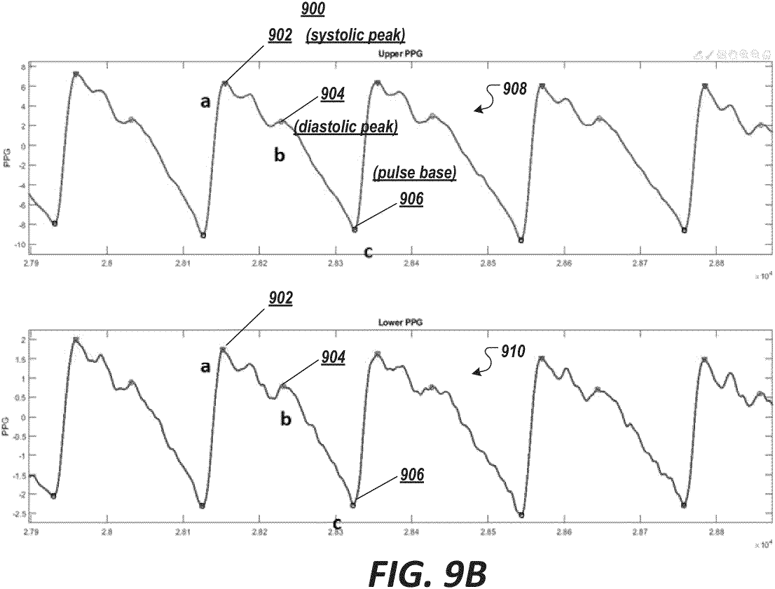

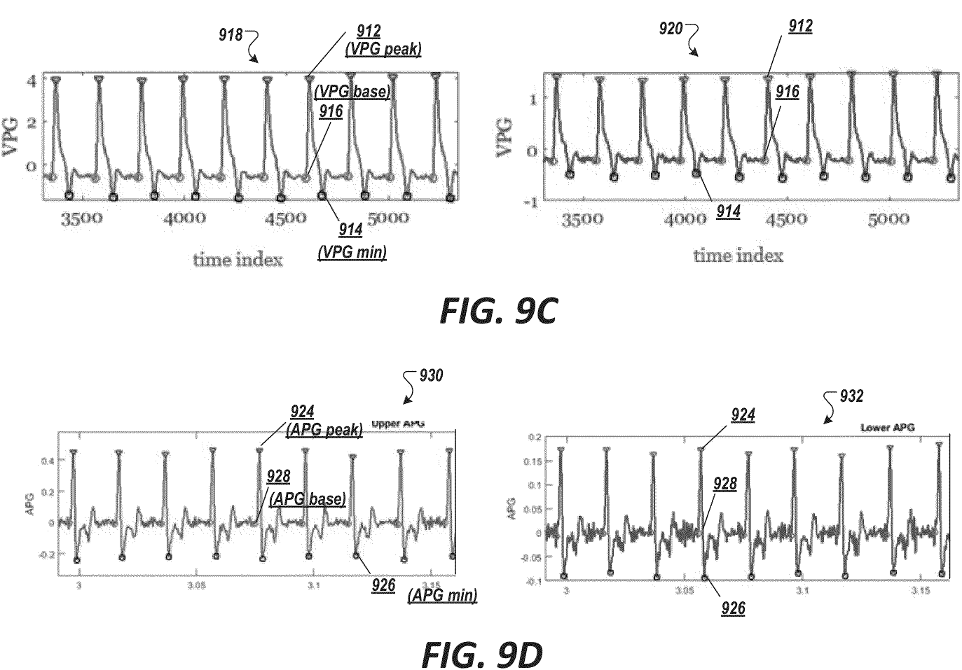

To determine (806) fiduciary landmarks in the PPG (absorptive), VPG, and APG waveforms, Module 400 employs a hemodynamic delineator that is configured to detect (i) landmarks or fiducial points such as pulse base, systolic peak, and diastolic peak landmarks in the PPG (absorptive) waveform and/or (ii) peak, minimum, and base landmarks in the VPG or APG waveforms. FIG. 9A shows an example conversion of the PPG waveform to an adsorptive PPG signal. FIG. 9B shows example pulse base landmarks (906), diastolic peak landmarks (904), and systolic peak landmarks (902) in a PPG waveform. FIG. 9C shows example VPG peak landmarks (912), VPG minimum landmarks (914), and VPG base landmarks (916) in a VPG waveform (shown as 918 and 920) that may be extracted by an exemplary hemodynamic delineator in accordance with an illustrative embodiment. FIG. 9D shows example APG peak landmarks (924), APG minimum landmarks (926), and APG base landmarks (928) in an AGP waveform (shown as 930 and 932) that may be extracted by an exemplary hemodynamic delineator in accordance with an illustrative embodiment.

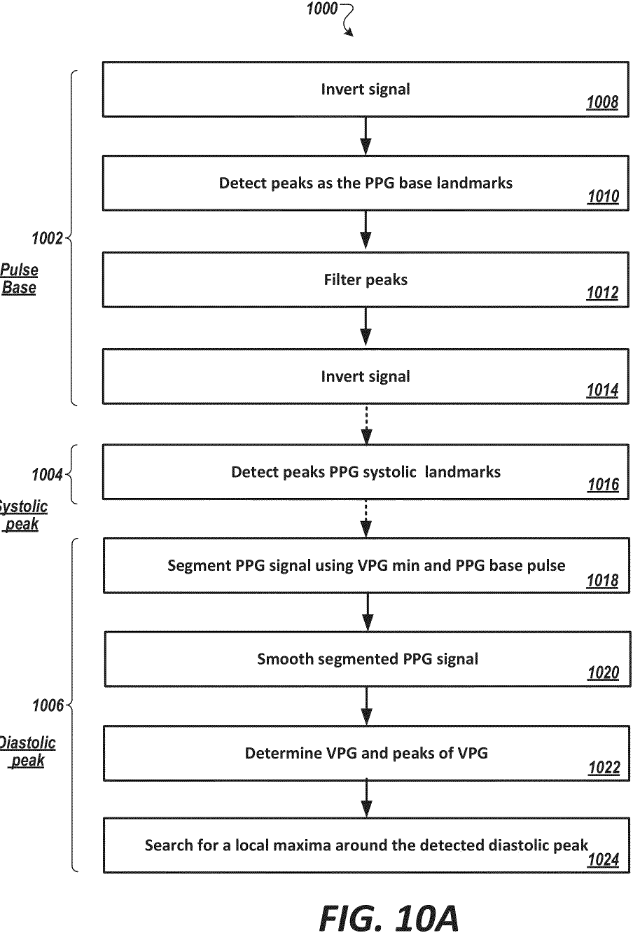

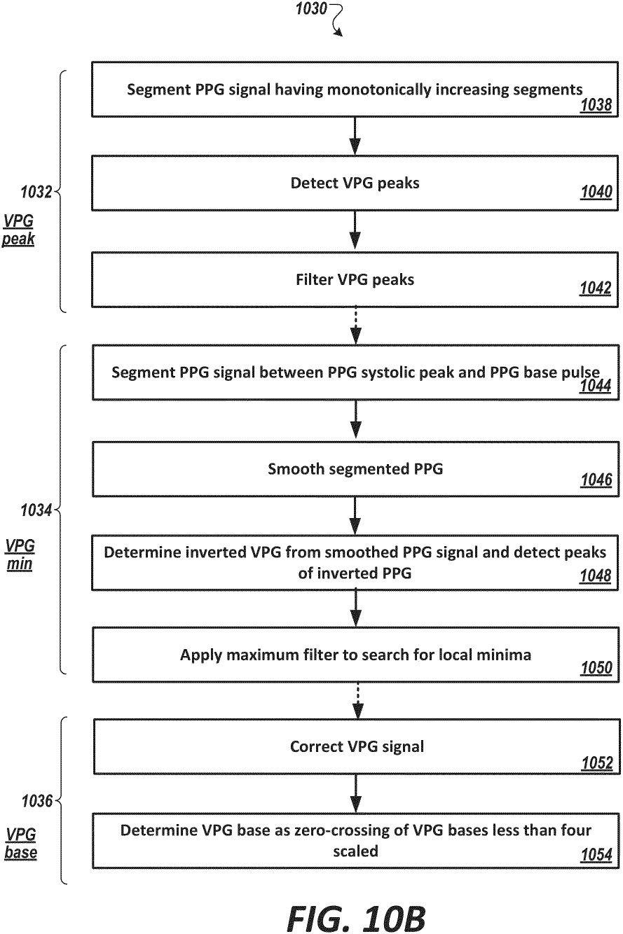

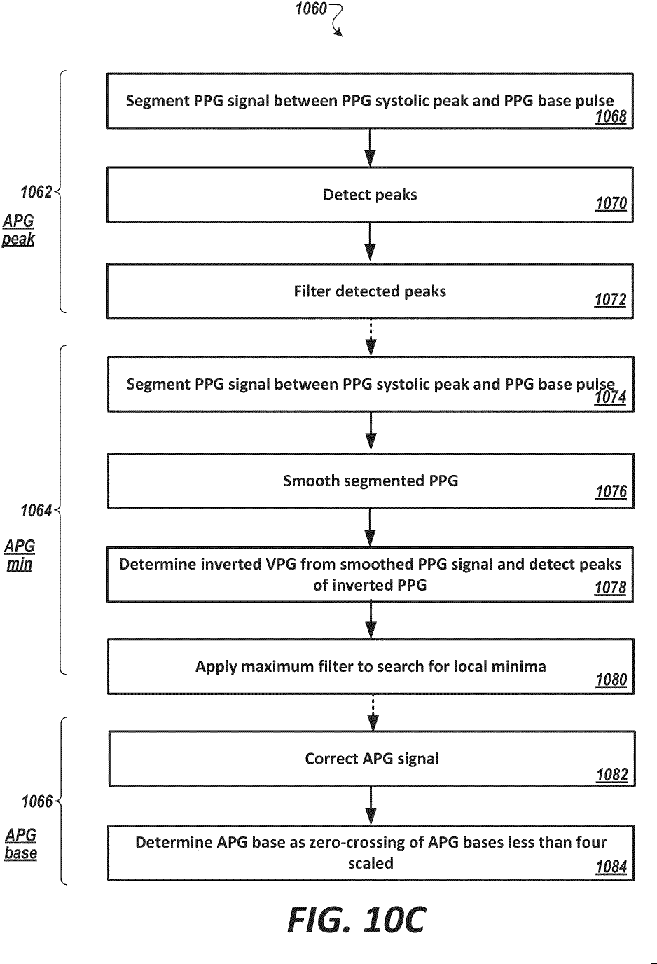

Hemodynamic Delineator. FIGS. 10A-10C each various aspects of the operation of a Hemodynamic Delineator to perform the method of FIG. 8A, among others, in accordance with an illustrative embodiment. FIG. 10A shows a method to hemodynamically delineate a PPG waveform. FIG. 10B shows a method to hemodynamically delineate a VPG waveform. FIG. 10C shows a method to hemodynamically delineate an APG waveform.

Photoplethysmographic (PPG) Waveform Segmentation. FIG. 10A is a diagram of an example method 1000 of Module 400 to hemodynamically delineate the fiduciary landmarks of a PPG waveform. In FIG. 10A, pulse base landmarks (906) can be detected as shown in steps 1002, systolic peak landmarks (902) can be detected as shown in steps 1004, and diastolic peak landmarks (904) can be detected as shown in steps 1006.

PPG Pulse Base (906). Referring to FIG. 10A, Method 1000 includes inverting (1008) the photoplethysmographic signal, so the pulse bases are presented as peaks in the inverted time series. The method 1000 can then detect peaks (1010). In some embodiments, a peak is defined as having a minimum pulse of 125 ms (e.g., equal to 25% of the minimum beat duration, 500 ms at the heart rate of 120 bpm). Method 1000 may then include filtering (1012) the detected peaks. In some embodiments, the filter (1012) may use the criteria: (i) the minimum peak width (at the half-prominence) having less than the minimum pulse width of 125 ms, (ii) pulse base values should always be smaller than zero (since the DC component of the signal is removed), (iii) and pulse base value should be less than ten scaled median absolute deviations (MAD) away from the median of the detected pulse bases. Method 1000 includes re-inverting (1014) the signal again back to its original position.

The pulse bases (906) may be used to isolate and create a 20-second segment of the signal containing a single cycle centered at the second 10 and padded with the base onset and offset. The PPG pulse base (906) may be detected and used for the detection of other landmarks.

Systolic peak (902): FIG. 10A also shows step 1016 to detect systolic peaks (902). The peaks (902) may be detected (1016) with a minimum pulse of 125 ms. The operation may be performed independent of the base pulse detection (e.g., before, following, or concurrent with the pulse base detection). The detected peaks may be filtered using the criteria: (i) the minimum peak width (at the half-prominence) being less than the minimum pulse width of 125 ms and (ii) systolic peak values being less than ten-scaled median absolute deviations (MAD) away from the median of the detected pulse bases. A maximum filter may be applied to detect the maximum values of the detected peaks within two consecutive pulse bases as the systolic peak for a corresponding cycle.

Diastolic peak (904): FIG. 10A also shows the method 1006 to detect PPG diastolic peak (904). Method 1006 includes segmenting (1018) the PPG signal using indexes of VPG min at cycle n and the consecutive PPG pulse base at cycle n+1. Method 1006 includes smoothing (1020) the segmented PPG using a smoothing operator (e.g., a 20-datapoint Gaussian-weighted moving average filter). Other averaging filters may be used. Method 1006 may include determining (1022) a VPG signal from the smoothed PPG signal and detecting (1022) peaks in the determined VPG signal. Method 1006 may include filtering (1024) the detected VPG peaks by application of a time constraint that the local maxima be at the first 50% of indexes of the segmented VPG. In some embodiments, operation 1024 may employ a maximum filter that is applied to the original PPG (non-smoothed) to search for local maxima around the detected diastolic peak in the smoothed PPG to determine the diastolic peak.

To determine the VPG min (914), the PPG signal is segmented using the indexes of PPG systolic peak (e.g., 902) at cycle n and the consecutive PPG pulse base (e.g., 906) at cycle n + 1. The segmented PPG is smoothened, e.g., with a 20-datapoint Gaussian-weighted moving average filter. A VPG signal is derived from the smoothed PPG, and the peaks for the inverted VPG are detected using a peak finder operator. The detected peaks are then filtered by applying a time constraint that the VPG min should occur at the first 30% of indexes of the segmented VPG. A maximum filter is applied to the original VPG (non-smoothed) that searches for local minima around the detected VPG min in the smoothed VPG.

Velocityplethysmogram (VPG) Waveform Segmentation. FIG. 10B is a diagram of an example method 1030 of Module 400 to hemodynamically delineate the fiduciary landmarks of a VPG waveform, as shown in FIG. 9C, in accordance with an illustrative embodiment. In FIG. 10B, VPG peak landmarks (912) can be detected as shown in step 1002, VPG minimum landmarks (914) can be detected as shown in step 1004, and VPG base landmarks (916) can be detected as shown in step 1006.

VPG Peak (912). To find the VPG peak landmarks (912), Method 1032 includes segmenting (1038) indexes from a PPG signal corresponding to a monotonically increasing segment in the PPG signal, also referred to as PPG raise-segments. The PPG raise-segments for cycle n may be identified as the data points between the PPG pulse base at cycle n and a consecutive PPG systolic peak at cycle n+1. Method 1032 may detect (1040) VPG peaks using a peak finder operator, e.g., configured with a minimum pulse width of 25% of the median of the PPG raise-duration (the time in ms associated with the PPG raise-segment) across the VPG signal. Method 1032 may filter (1042) the detected peaks using the criteria: (i) the minimum peak width (at the half-prominence) be less than the VPG minimum pulse width and (ii) VPG peak values be less than ten scaled MAD away from the median of the detected peaks. Method 1032 may apply (1042) a maximum filter to identify the maximum value of the detected peaks within the PPG raised segment as the VPG peak for the corresponding cycle.

VPG min. To determine the VPG min (912), Method 1034 includes segmenting (1044) the PPG signal using the indexes of the PPG systolic peak (e.g., 902) at cycle n and the consecutive PPG pulse base at cycle n+1. Method 1034 includes smoothing (1046) the segmented PPG, e.g., with a 20-datapoint Gaussian-weighted moving average filter. Method 1034 may derive (1048) an inverted VPG signal from the smoothed PPG and detect (1048) peaks of the inverted VPG. To detect the peaks, Method 1034 may filter for detected peaks having a time constraint that the VPG min occurs at the first 30% of indexes of the segmented VPG. Method 1034 may apply (1050) a maximum filter to the original VPG (non-smoothed) to search for local minima around the detected VPG min in the smoothed VPG.

VPG base (916). To determine the VPG base (916), Method 1036 includes correcting (1052) a baseline from the VPG signal that may occur during the numerical derivative operation performed on the PPG signal. The baseline correction (1052) may be performed by percentile filtering the VPG waveform to identify the data points with the values between the 20% and 50% percentile of the VPG waveform and then subtracting the VPF waveform from the mean of the percentile filtered VPG. Then, Method 1036 may complete the baseline correction 1052 by segmenting the baseline-corrected VPG using the indexes of VPG min (914) at cycle n and a consecutive VPG peak at cycle n+1. Once the baseline correction is completed, Method 1036 may determine (1054) the VPG bases through zero-crossing the VPG where VPG bases less than four-scaled median absolute deviations (MAD) away from the median of the detected bases may be removed.

Accelerationplethysmogram (APG) Waveform Segmentation. As noted above, FIG. 10C is a diagram of an example method 1060 of Module 400 to hemodynamically delineate the fiduciary landmarks of an APG waveform, as shown in FIG. 9C, in accordance with an illustrative embodiment. In FIG. 10C, APG peak landmarks (924) can be detected as shown in steps 1062, APG minimum landmarks (926) can be detected as shown in steps 1064, and APG base landmarks (928) can be detected as shown in steps 1066.

APG peak (924). To determine the APG peak (e.g., 924), Method 1062 includes segmenting (1068) the PPG waveform using the indexes PPG pulse base (e.g., 906) at cycle n and the consecutive PPG systolic peak (e.g., 902) at cycle n+1. Method 1062 then includes detecting (1070) the AGP peaks (924). Any peak detection algorithm may be used. An example is the “findpeak” function manufactured by MathWorks in Matlab R2019a. A maximum filter is applied (1072) so that the maximum value of the detected peaks within the segment is found, and peaks with the value of more than six scaled MAD away from the median of the detected may be removed.

APG min (926). To determine the APG min (e.g., 926), Method 1064 includes segmenting (1074) the PPG signal using the indexes of the PPG pulse base (e.g., 906) at cycle n and the consecutive PPG systolic peak (e.g., 902) at cycle n+1. Method 1064 includes smoothing (1076) the segmented PPG. Any smoothing operator may be used. An example is a smooth-data operator manufactured by MathWorks in Matlab R2019a. The smoothing operator may employ a 20-datapoint Gaussian-weighted moving average filter. Method 1064 may derive (1078) an inverted APG signal from the smoothed PPG and detect (1078) peaks of the inverted APG. To detect the peaks, Method 1064 may filter for detected peaks having a time constraint that the APG min occurs at the first 75% of indexes of the segmented APG. Method 1064 then applies (1080) minimum filter to find the minimum value in the original APG (non-smoothed) by searching around the detected minimum in the smoothed APG. APG min with the value of more than six scaled MAD away from the median of the detected may be removed.

APG base (928). To determine the APG base (928), Method 1066 includes correcting (1082) a baseline from the APG signal that may occur during the numerical derivative operation performed on the PPG signal. The baseline correction (1082) may be performed by percentile filtering the APG waveform to identify the data points with the values between the 25% and 75% percentile of the APG waveform and then subtracting the APG waveform from the mean of the percentile filtered APG. Then, Method 1066 may complete the baseline correction 1082 by segmenting the baseline-corrected APG using the indexes of APG min (926) at cycle n and a consecutive APG peak at cycle n+1. Once the baseline correction is completed, Method 1066 may determine (1084) the APG bases through zero-crossing the VPG where VPG bases less than four-scaled median absolute deviations (MAD) away from the median of the detected bases may be removed.

### Example #2 - PPG, VPG, APG Duration Features

FIG. 5 illustrates, as the second of four example feature or parameter categories, an example PPG duration analysis feature computation module 500 configured to determine statistical attributes (e.g., mean, standard deviation, maximum, minimum, and/or min-max) of duration values of fiduciary points (e.g., peak, base, minimum) across multiple cycles of a PPG, VPG, and APG waveform. FIG. 8A show an example method of Module 500.

Table 3 shows an example set of 12 extractable waveform duration features and their corresponding description to provide up to 36 features or parameters.

Table 3

| | |
|---|---|
| Signal Type | Duration Feature |
| PPG | Diastolic peak-to-peak duration ("diasDist") Systolic peak-to-peak duration ("systDist) Pulse-base to pulse-base duration ("baseDist") Systolic-peak to diastolic peak duration ("diasDist_left") Systolic-to-pulse base duration ("systDist left") pulse-base to systolic peak duration ("pulseDist_left") |
| VPG or APG | Peak-to-peak duration ("peakDist") Min-to-min duration ("minDist) Base-to-base duration ("baseDist") Peak-to-base duration ("baseDist_left") Peak-to-minimum duration ("minDist_left") Minimum-to-peak duration ("peakDist left") |

Table 4 shows a set of 12 PPG waveform-based features for two PPG waveforms (from 2 PPG measurement channels) and an additional 24 features for the corresponding set of VPG and APG waveforms. In Table 4, 3 features (see “*” in Table 4) have been observed to have significant utility in the assessment of the presence or non-presence of at least one cardiac disease or condition - specifically, the determination of the presence or non-presence of elevated LVEDP. It has also been observed through experimentation that 3 feature types (see “**” in Table 4) have significant utility in the assessment of the presence or non-presence of coronary artery disease. The list of the specific features determined to have significant utility in the assessment of the presence or non-presence of abnormal or elevated LVEDP and the presence or non-presence of significant CAD is provided in Table 9B and Table 10B, respectively.

Table 4

Duration Features

| | | |
|---|---|---|
| PPG | VPG | APG |
| upPPG_mean_baseDist | upVPGmean_peakDist | upAPG_mean_peakDist |
| upPPG_mean_systDist | upVPG_mean_minDist | upAPG_mean_minDist* |
| upPPG_mean_diasDist | upVPG_mean_baseDist | upAPG_mean_baseDist |
| upPPG_mean_systDist_left** | upVPG_mean_minDist_left | upAPG_mean_minDist_left |
| upPPG_mean_diasDist_left** | upVPG_mean_baseDist_left | upAPG_mean_baseDist_left |
| upPPG_mean_pulseDist_left* | upVPG_mean_peakDist_left | upAPG_mean_peakDist_left |
| lowPPG_mean_baseDist | lowVPG_mean_peakDist | lowAPG_mean_peakDist |
| lowPPG_mean_systDist | lowVPG_mean_minDist | lowAPG_mean_minDist |
| lowPPG_mean_diasDist | lowVPG_mean_baseDist | lowAPG_mean_baseDist |
| lowPPG_mean_systDist_left | lowVPG_mean_minDist_left* | lowAPG_mean_minDist_left |
| lowPPG_mean_diasDist_left** | lowVPG_mean_baseDist_left | lowAPG_mean_baseDist_left |
| lowPPG_mean_pulseDist_left | lowVPG_mean_peakDist_left | lowAPG_mean_peakDist_left |

FIG. 8A also shows a method 800 to generate PPG waveform-based features or parameters, e.g., as performed by the PPG duration feature computation module 400 of FIG. 4, in accordance with an illustrative embodiment, which can be used wholly, or partially, to generate PPG waveform-based features or parameters and its outputs to be used in machine-learned classifier to determine a metric associated with a physiological system of a subject under study. To determine the features (median, standard deviation, kurtosis, minimum, maximum, min-max), a distribution of duration attributes calculated from cycles in the delineated PPG, VPG, and APG waveforms is determined.

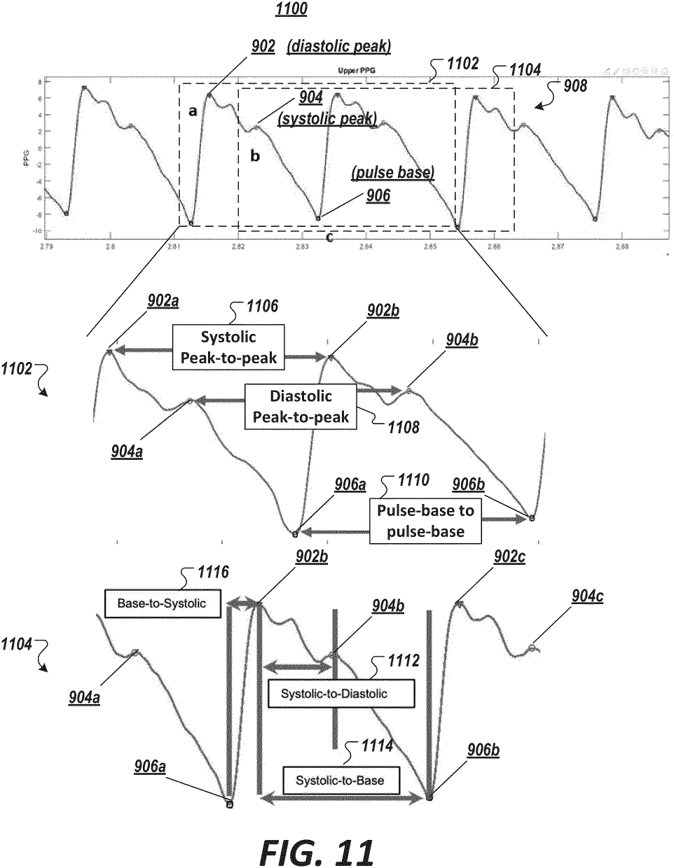

FIG. 11 shows example duration-associated features defined between like fiduciary landmarks across multiple cycles (also referred to as beat-to-beat duration features or inter-beat duration features) in accordance with an illustrative embodiment. In FIG. 11, the PPG waveform 908 of FIG. 9B is shown with window 1102 and window 1104. In window 1102, a systolic peak-to-peak duration 1106 is defined between systolic peak n (shown as 902a) and next systolic peak n+1 (shown as 902b) for a PPG waveform (also referred to as PPG systolic peak-to-peak duration “diasDist”). A diastolic peak-to-peak duration 1106 is also defined between diastolic peak n (shown as 904a) and next diastolic peak n+1 (shown as 904b). A pulse-based peak-to-peak duration 1110 is defined between pulse base n (shown as 906a) and a next pulse base n+1 (shown as 906b) (also referred to as PPG diastolic peak-to-peak duration “diasDist” and PPG pulse-base to pulse-base duration).

In window 1104 of FIG. 11, a systolic peak to diastolic peak duration 1112 is defined between systolic peak n (shown as 902b) and diastolic peak n (shown as 904b) for a PPG waveform (also referred to as systolic-to-diastolic duration “diasDist_left”). A systolic peak to pulse base duration 1114 is defined between systolic peak n (shown as 902b) and the pulse base n (shown as 906b) (also referred to as systolic-peak to pulse-base duration “pulseDist_left.” A pulse-base to systolic peak duration 1116 is defined between pulse base n-1 (shown as 906a) and the systolic peak n (shown as 902b) (also referred to as systolic-peak to pulse-base duration “systolicDist_left” and pulse-base to systolic peak duration “baseDist_left,” respectively).

A corresponding set of intra-beat duration features can be defined between peaks, minimums, and base landmarks of a VPG waveform and an APG waveform (shown in Table 5 as peak-to-base duration (“baseDist_left”), peak-to-minimum duration (“minDist_left”), and minimum-to-peak duration (“peakDist_left”) for VPG and APG waveforms.

### Example #3 - Waveform Geometry Features

FIG. 6 illustrates, as the third of four example feature or parameter categories, an example PPG geometric analysis feature computation module 600 configured to determine statistical attributes (e.g., mean, standard deviation, maximum, minimum, and/or min-max) of topologic values (e.g., angles) of a polygon defined among fiduciary points (e.g., peak, base, minimum) across multiple cycles of a PPG, VPG, and APG waveform. FIG. 8B, discussed below, shows an example method of Module 400.

Table 5 shows an example set of 4 types of extractable waveform topologic features and their corresponding description to provide up to 12 features or parameters.

Table 5

| | |
|---|---|
| Signal Type | Geometric Feature |
| PPG | Tri_P1angle |
| VPG | Tri P2angle |
| APG | Tri P3angle |
| | Tri area |

Table 6 shows a set of 4 PPG waveform-based features for a PPG waveform and an additional 8 features for the corresponding set of VPG and APG waveforms. In Table 6, all 12 features (see “*” in Table 6) have been observed to have significant utility in the assessment of the presence or non-presence of at least one cardiac disease or condition - specifically, the determination of presence or non-presence of elevated LVEDP. It has also been observed through experimentation that 6 feature types (see “**” in Table 4) have significant utility in the assessment of the presence or non-presence of coronary artery disease. The list of the specific features determined to have significant utility in the assessment of the presence or non-presence of abnormal or elevated LVEDP and the presence or non-presence of significant CAD is provided in Table 9C and Table 10C, respectively.

Table 6

Waveform Geometric Features

| | | |
|---|---|---|
| PPG | VPG | APG |
| upPPG_Tri1_P 1 angle* | upPPG_Tri2_P1angle*’** | upPPG_Tri3_P1angle* |
| upPPG_Tri1_P2angle*'** | upPPG_Tri2_P2angle* | upPPG_Tri3_P2angle* |
| upPPG_Tri1_P3angle*'** | upPPG_Tri2_P3angle*’** | upPPG_Tri3_P3angle* |
| upPPG_Tri1_area*'** | upPPG_Tri2_area*'** | upPPG_Tri3_area* |

FIG. 8B also shows a method 810 to generate PPG waveform-based features or parameters, e.g., as performed by the PPG geometric feature computation module 600 of FIG. 6, in accordance with an illustrative embodiment, which can be used wholly, or partially, to generate PPG waveform-based features or parameters and its outputs to be used in machine-learned classifier to determine a metric associated with a physiological system of a subject under study. To determine the features of Table 6, Module 600 is configured, in some embodiments, to (i) pre-process (802) the acquired PPG signal, (ii) generate (804) a VPG and APG waveforms from the PPG signal, (iii) determine (806) fiduciary landmarks in the PPG, VPG, and APG waveforms. Module 600 can then (iv) determine (810) a triangle object among the fiduciary landmarks and (v) extract (812) features or parameters using the triangle object.

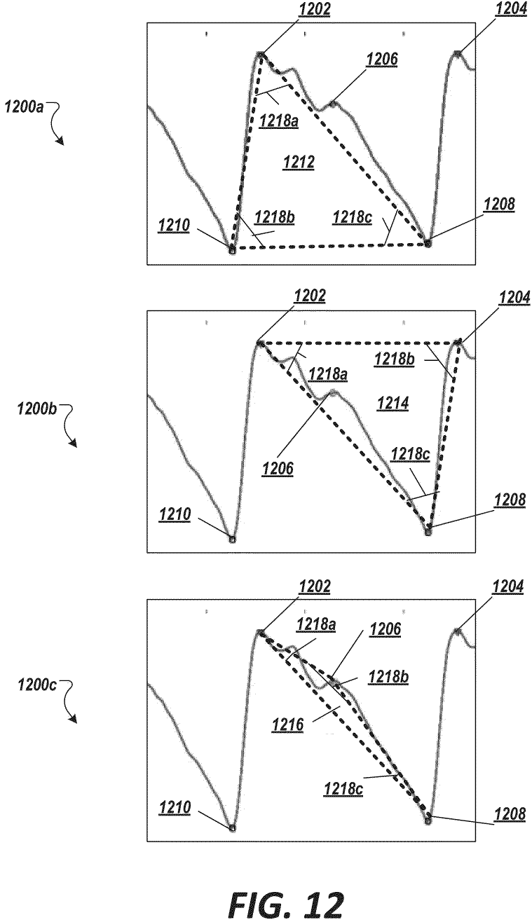

FIG. 12 shows an example waveform geometry model, comprising triangle models, defined among a set of determined fiduciary landmarks in a PPG waveform in accordance with an illustrative embodiment. In FIG. 12, a representative PPG waveform 1200 (shown as 1200a, 1200b, and 1200b) is generated. The representative PPG waveform 1200 includes an average systolic peak 1202, an average nearby systolic peak 1204, an average diastolic peak 1206, an average pulse base 1208, and an average nearby pulse base 1210 (the waveform is shown only for illustrative purposes). Within FIG. 12, three triangles 1212, 1214, and 1216 are generated within the representative waveform (shown as 1200a-1200c).

In the waveform associated with 1200a, the first triangle 1212 is defined among the average systolic peak 1202, the average pulse base 1208, and the average nearby pulse base 1210. In the waveform associated with 1200b, the second triangle 1214 is defined among the average systolic peak 1202, the average nearby systolic peak 1204, and the average pulse base 1208. In the waveform associated with 1200c, the third triangle 1216 is defined among the average systolic peak 1202, the average diastolic peak 1206, and the average pulse base 1208.

For each of the triangles (e.g., 1212, 1214, 1216), the angles (shown as 1218a, 1218b, 1218c) and the area of the respective triangle may be determined.

### Example #4 - PPG-Based SpO₂ Features or Parameters

FIG. 7 illustrates, as the fourth of four example feature or parameter categories, an example PPG SpO₂ analysis feature computation module 700 configured to determine attributes associated with the measure of oxygen level in the blood.

Oxygen saturation level (SpO₂) is the measure of oxygen level in the blood given by Equation 1.

$$S p {O}_{2} \left(\%\right) = \frac{H b {O}_{2}}{H b {O}_{2} + H b} \times 100$$

In Equation 1, HbO₂ and Hb are oxyhemoglobin and deoxyhemoglobin values in the blood, respectively. The ratio in Equation 1 may be acquired using a pulse oximeter by extracting the AC and DC components of two photoplethysmographic waveforms. Oxygen saturation level may be calculated per Equation 2, where K is a constant proportional factor and R is defined per Equation 3.

$$S p {O}_{2} \left(\%\right) = K \times R$$

$$R = \frac{\frac{A {C}_{R}}{D {C}_{R}}}{\frac{A {C}_{I R}}{D {C}_{I R}}}$$

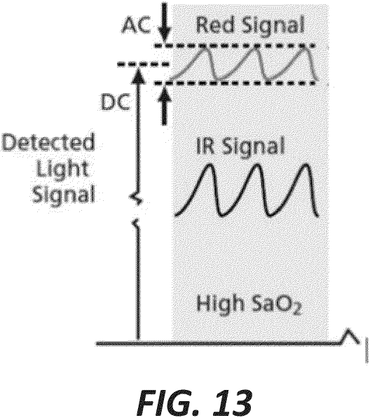

FIG. 13 shows the AC and DC components of two photoplethysmographic waveforms used to generate SpO₂ features in accordance with an illustrative embodiment. Specifically, FIG. 13 shows the AC and DC components of two photoplethysmographic waveforms (labeled as “Red Signal” and “IR Signal”). Table 7 provides a summarized list of SpO₂ features that are extractable from two or more photoplethysmographic waveforms and their respective corresponding description.

It has also been observed through experimentation that 8 feature types (see “**” in Table 4) have significant utility in the assessment of the presence or non-presence of coronary artery disease. The list of the specific features determined to have significant utility in the assessment of the presence or non-presence of significant CAD is provided in Table 10D.

Table 7

| | |
|---|---|
| Feature Name | Description |
| median_rSpo2_num** | R * 100,000 |
| median_rSpo2_vec** | R is calculated at every point in the signal, and the median value of R vector is returned. |
| median_rDenominator_num** | Infrared AC/DC. Denominator value of R formula |
| median_rNumerator_num** | Red AC/DC. Numerator value of R formula |
| rSpo2_std** | The standard deviation of the R vector |
| rSpo2_max** | Max value of the R vector |
| rSpo2_min** | Min value of the R vector |
| rSpo2_minMaxRatio** | Min/Max ratio of the R vector |
| rSpo2_kurt | Kurtosis of the R vector |
| rSpo2_skew | Skewness of the R vector |

Other SpO₂-associated features may be generated, including, for example, per the description provided in Cho et al., “A preliminary study on photoplethysmogram (PPG) signal analysis for reduction of motion artifact in frequency domain,” 2012 IEEE-EMBS Conference on Biomedical Engineering and Sciences, Langkawi, pp. 28-33 (2012).

### Experimental Results and Examples

Several development studies have been conducted to develop feature sets, and in turn, algorithms that can be used to estimate the presence or non-presence, severity, or localization of diseases, medical conditions, or an indication of either. In one study, algorithms were developed for the non-invasive assessment of abnormal or elevated LVEDP. As noted above, abnormal or elevated LVEDP is an indicator of heart failure in its various forms. In another development study, algorithms and features were developed for the non-invasive assessment of coronary artery disease.

As part of these two development studies, clinical data were collected from adult human patients using a biophysical signal capture system and according to protocols described in relation to FIG. 2. The subjects underwent cardiac catheterization (the current “gold standard” tests for CAD and abnormal LVEDP evaluation) following the signal acquisition, and the catheterization results were evaluated for CAD labels and elevated LVEDP values. The collected data were stratified into separate cohorts: one for feature/algorithm development and the other for their validation.

Within the feature development phases, features were developed, including the PPG waveform-based features or parameters, to extract characteristics in an analytical framework from biopotential signals (as an example of the cardiac signals discussed herein) and photo-absorption signals (as examples of the hemodynamic or photoplethysmographic discussed herein) that are intended to represent properties of the cardiovascular system. Corresponding classifiers were also developed using classifier models, linear models (e.g., Elastic Net), decision tree models (XGB Classifier, random forest models, etc.), support vector machine models, and neural network models to non-invasively estimate the presence of an elevated or abnormal LVEDP. Univariate feature selection assessments and cross-validation operations were performed to identify features for use in machine learning models (e.g., classifiers) for the specific disease indication of interest. Further description of the machine learning training and assessment are described in a U.S. provisional patent application concurrently filed herewith entitled “Method and System to Non-Invasively Assess Elevated Left Ventricular End-Diastolic Pressure” having attorney docket no. 10321-048pv1, which is hereby incorporated by reference herein in its entirety.

The univariate feature selection assessments evaluated many scenarios, each defined by a negative and a positive dataset pair using a t-test, mutual information, and AUC-ROC evaluation. The t-test is a statistical test that can determine if there is a difference between two sample means from two populations with unknown variances. Here, the t-tests were conducted against a null hypothesis that there is no difference between the means of the feature in these groups, e.g., normal LVEDP vs. elevated (for LVEDP algorithm development); CAD- vs. CAD+ (for CAD algorithm development). A small p-value (e.g., ≤ 0.05) indicates strong evidence against the null hypothesis.

Mutual information (MI) operations were conducted to assess the dependence of elevated or abnormal LVEDP or significant coronary artery disease on certain features. An MI score greater than one indicates a higher dependency between the variables being evaluated. MI scores less than one indicates a lower dependency of such variables, and an MI score of zero indicates no such dependency.

A receiver operating characteristic curve, or ROC curve, illustrates the diagnostic ability of a binary classifier system as its discrimination threshold is varied. The ROC curve may be created by plotting the true positive rate (TPR) against the false positive rate (FPR) at various threshold settings. AUC-ROC quantifies the area under a receiver operating characteristic (ROC) curve - the larger this area, the more diagnostically useful the model is. The ROC, and AUC-ROC, value is considered statistically significant when the bottom end of the 95% confidence interval is greater than 0.50.

Table 8 shows an example list of the negative and the positive dataset pair used in the univariate feature selection assessments. Specifically, Table 8 shows positive datasets being defined as having an LVEDP measurement greater than 20 mmHg or 25 mmHg, and negative datasets were defined as having an LVEDP measurement less than 12 mmHg or belonging to a subject group determined to have normal LVEDP readings.

Table 8

| | |
|---|---|
| Negative Dataset | Positive Dataset |
| ≤ 12 (mmHg) | ≥ 20 (mmHg) |
| ≤ 12 (mmHg) | ≥ 25 (mmHg) |
| Normal LVEDP | ≥ 20 (mmHg) |
| Normal LVEDP | ≥ 25 (mmHg) |

Tables 9A, 9B, 9C each shows a list of PPG waveform-based features having been determined to have utility in estimating the presence and non-presence of elevated LVEDP in an algorithm executing in a clinical evaluation system. The features of Tables 9A, 9B, and 9C and corresponding classifiers have been validated to have clinical performance comparable to the gold standard invasive method to measure elevated LVEDP.

Table 9A

| Features | t-test | AUC | MI |
|---|---|---|---|
| upPPG mean pulseBase | 0.0403 | n/s | n/s |
| lowPPG mean pulseBase | n/s | 0.5057 | n/s |
| upPPG max pulseBase | 0.0415 | n/s | n/s |
| lowPPG max pulseBase | 0.0485 | 0.5052 | n/s |
| lowPPG min pulseBase* | 0.0315 | n/s | n/s |
| upPPG mean systPeak | 0.0154 | n/s | n/s |
| lowPPG mean systPeak | 0.0274 | 0.5046 | n/s |
| upPPG min systPeak | 0.0173 | n/s | n/s |
| lowPPG min systPeak | 0.0278 | 0.5163 | n/s |
| lowPPG std systPeak | n/s | 0.5020 | n/s |
| upPPG max systPeak | 0.0218 | n/s | n/s |
| lowPPG max systPeak | 0.0379 | 0.5120 | n/s |
| upVPG mean base | 0.0293 | n/s | n/s |
| lowVPG mean base | 0.0382 | 0.5115 | n/s |
| upVPG max base | 0.0300 | n/s | n/s |
| lowVPG max base | 0.0224 | 0.5124 | n/s |
| upVPG min base | 0.0311 | n/s | n/s |
| lowVPG min base | n/s | 0.5071 | n/s |
| upVPG minMax base | n/s | 0.5107 | n/s |
| lowVPG minMax base* | n/s | 0.5174 | n/s |
| lowVPG mean peak | 0.0454 | n/s | n/s |
| upVPG max peak | 0.0475 | n/s | n/s |
| lowVPG max peak* | 0.0153 | n/s | n/s |
| lowVPG min peak | 0.0361 | n/s | n/s |
| lowVPG mean min | 0.0453 | 0.5090 | n/s |
| upVPG max min | 0.0221 | n/s | n/s |
| lowVPG max min | 0.0284 | 0.5012 | n/s |
| upVPG min min | 0.0257 | n/s | n/s |
| lowVPG min min | n/s | 0.5144 | n/s |
| upAPG mean base | 0.0230 | 0.5246 | n/s |
| upAPG mean peak | 0.0140 | 0.5053 | n/s |
| lowAPG max peak* | n/s | 0.5001 | 1.2860 |
| lowAPG mean base* | 0.0363 | n/s | n/s |

LVEDP <= 12 (N=246) vs >=20 (N=209) * LVEDP <= 12 (N=246) vs >=25 (N=78)

Table 9B

| Features | t-test | AUC | MI |
|---|---|---|---|
| lowAPG mean minDist* | 0.0256 | n/s | n/s |
| lowPPG mean pulseDist left* | 0.0485 | 0.5062 | n/s |
| lowVPG mean minDist left | 0.0475 | n/s | n/s |

LVEDP <= 12 (N=246) vs >=20 (N=209) * LVEDP <= 12 (N=246) vs >=25 (N=78)

Table 9C

| Features | t-test | AUC | MI |
|---|---|---|---|
| upPPG_Tri1_area | n/s | 0.5053 | n/s |
| upPPG_Tri1_P1angle | 0.0250 | n/s | n/s |
| upPPG_Tri1_P2angle | 0.0129 | n/s | n/s |
| upPPG_Tri1_P3angle | 0.0181 | n/s | n/s |
| upPPG_Tri2_area | n/s | 0.5022 | n/s |
| upPPG_Tri2_P1angle | 0.0178 | n/s | n/s |
| upPPG_Tri2_P2angle | 0.0244 | n/s | n/s |
| upPPG_Tri2_P3angle | 0.0129 | 0.5076 | n/s |
| upPPG_Tri3_area | 0.0320 | n/s | n/s |
| upPPG_Tri3_P1angle | 0.0178 | n/s | n/s |
| upPPG Tri3 P2angle | 0.0152 | n/s | n/s |
| upPPG Tri3 P3angle | 0.0206 | n/s | n/s |

LVEDP <= 12 (N=246) vs >=20 (N=209)

Tables 10A, 10B, 10C, and 10D each shows a list of PPG waveform-based features having been determined to have utility in estimating the presence and non-presence of significant CAD in an algorithm executing in a clinical evaluation system. The features of Tables 10A, 10B, 10C, and 10D and corresponding classifiers have been validated to have clinical performance comparable to the gold standard invasive method to measure significant CAD.

Table 10A

| Features | t-test | AUC | MI |
|---|---|---|---|
| lowPPG max diasPeak | 0.0131 | n/s | 1.4395 |
| upPPG max diasPeak | 0.0119 | 0.5143 | n/s |
| lowPPG max pulseBase | 0.0100 | n/s | n/s |
| upPPG max pulseBase | 0.0088 | n/s | n/s |
| lowPPG max systPeak | 0.0085 | n/s | n/s |
| upPPG max systPeak | 0.0092 | 0.5034 | 1.2153 |
| upPPG mean diasPeak | n/s | 0.5022 | n/s |
| lowPPG mean pulseBase | 0.0079 | n/s | n/s |
| upPPG mean pulseBase | 0.0075 | 0.5165 | n/s |
| lowPPG mean systPeak | 0.0120 | n/s | n/s |
| upPPG mean systPeak | 0.0094 | 0.5070 | 1.6068 |
| lowPPG min pulseBase | 0.0058 | n/s | 1.0464 |
| upPPG min pulseBase | 0.0069 | 0.5069 | n/s |
| lowPPG min systPeak | 0.0148 | 0.5149 | n/s |
| upPPG min systPeak | 0.0099 | n/s | n/s |
| lowPPG minMax pulseBase | 0.0415 | n/s | n/s |
| lowPPG std diasPeak | 0.0008 | n/s | n/s |
| upPPG std diasPeak | 0.0013 | n/s | n/s |
| lowPPG std pulseBase | 0.0338 | n/s | n/s |
| lowPPG std systPeak | 0.0330 | n/s | n/s |
| lowVPG max base | 0.0047 | n/s | n/s |
| upVPG max base | 0.0049 | n/s | n/s |
| lowVPG max min | 0.0316 | n/s | n/s |
| upVPG max min | 0.0152 | n/s | n/s |
| lowVPG mean base | 0.0053 | n/s | n/s |
| upVPG mean base | 0.0058 | n/s | n/s |
| lowVPG mean min | 0.0443 | n/s | n/s |
| upVPG mean min | 0.0228 | n/s | n/s |
| lowVPG min base | 0.0058 | n/s | n/s |
| upVPG min base | 0.0060 | n/s | n/s |
| lowVPG min min | 0.0287 | n/s | n/s |
| upVPG min min | 0.0270 | n/s | n/s |
| lowVPG minMax base | n/s | n/s | 1.5106 |
| lowVPG minMax min | n/s | 0.5041 | 1.6128 |
| lowVPG std peak | 0.0473 | n/s | n/s |
| upVPG std peak | n/s | n/s | 1.0599 |
| lowAPG max peak | n/s | 0.5029 | n/s |
| upAPG max peak | 0.0493 | n/s | n/s |
| lowAPG mean base | 0.0378 | n/s | n/s |
| upAPG mean base | 0.0077 | n/s | n/s |
| lowAPG mean minDist left | n/s | n/s | 1.2861 |
| upAPG mean minDist left | n/s | n/s | 1.0493 |
| lowAPG mean peak | n/s | 0.5020 | n/s |
| upAPG mean peak | 0.0381 | n/s | n/s |
| lowAPG min peak | n/s | 0.5130 | n/s |
| upAPG min peak | 0.0206 | 0.5085 | n/s |
| lowAPG minMax min | n/s | n/s | 1.0878 |
| lowAPG minMax peak | 0.0105 | 0.5110 | n/s |
| upAPG minMax peak | 0.0013 | 0.5255 | n/s |

FA scenario = significant CAD (e.g., defined as > 70% blockage and/or FFR < 0.8) (N = 464; 232 CAD positives and 232 CAD negatives (½ single and ½ multi-vessel disease) (½ are males and ½ are females)

Table 10B

| Features | t-test | AUC | MI |
|---|---|---|---|
| lowPPG mean diasDist left | n/s | 0.5098 | n/s |
| upPPG mean diasDist left | n/s | 0.5189 | n/s |
| upPPG mean systDist left | n/s | n/s | 1.0362 |
| median rSpo2 vec | 0.0404 | n/s | n/s |
| median rDenominator num | n/s | 0.5236 | n/s |
| median rNumerator num | n/s | n/s | 1.2249 |

FA scenario = significant CAD (e.g., defined as > 70% blockage and/or FFR < 0.8) (N = 464; 232 CAD positives and 232 CAD negatives (½ single and ½ multi-vessel disease) (½ are males and ½ are females)

Table 10C

| Features | t-test | AUC | MI |
|---|---|---|---|
| upPPG Tri1_area | 0.0143 | n/s | n/s |
| upPPG Tri1_P2angle | 0.0043 | n/s | n/s |
| upPPG Tri1_P3angle | 0.0256 | n/s | n/s |
| upPPG Tri2 area | 0.0145 | n/s | n/s |
| upPPG Tri2_P1angle | 0.0246 | n/s | n/s |
| upPPG Tri2 P3angle | 0.0042 | n/s | n/s |

FA scenario = significant CAD (e.g., defined as > 70% blockage and/or FFR < 0.8) (N = 464; 232 CAD positives and 232 CAD negatives (½ single and ½ multi-vessel disease) (½ are males and ½ are females)

Table 10D

| Features | t-test | AUC | MI |
|---|---|---|---|
| rSpo2 max | 0.0035 | n/s | 1.4597 |
| rSpo2 min | 0.0277 | n/s | 1.8822 |
| rSpo2 minMaxRatio | 0.0036 | 0.5142 | 1.6172 |
| rSpo2 std | 0.0077 | 0.5131 | 1.1774 |
| median rSpo2 num | n/s | 0.5179 | n/s |

FA scenario = significant CAD (e.g., defined as > 70% blockage and/or FFR < 0.8) (N = 464; 232 CAD positives and 232 CAD negatives (½ single and ½ multi-vessel disease) (½ are males and ½ are females)

The determination that certain PPG waveform-based features have clinical utility in estimating the presence and non-presence of elevated LVEDP or the presence and non-presence of significant CAD provides a basis for the use of these PPG waveform-based features or parameters, as well as other features described herein, in estimating for the presence or non-presence and/or severity and/or localization of other diseases, medical condition, or an indication of either particularly, though not limited to, heart disease or conditions described herein.

The experimental results further indicate that intermediary data or parameters of PPG waveform-based features also have clinical utility in diagnostics as well as treatment, controls, monitoring, and tracking applications.

### Example Clinical Evaluation System

FIG. 14A shows an example clinical evaluation system 1400 (also referred to as a clinical and diagnostic system) that implements the modules of FIG. 1 to non-invasively compute PPG waveform-based features or parameters, along with other features or parameters, to generate, via a classifier (e.g., machine-learned classifier), one or more metrics associated with the physiological state of a patient or subject according to an embodiment. Indeed, the feature modules (e.g., of FIGS. 1, 4-14) can be generally viewed as a part of a system (e.g., the clinical evaluation system 1400) in which any number and/or types of features may be utilized for a disease state, medical condition, an indication of either, or combination thereof that is of interest, e.g., with different embodiments having different configurations of feature modules. This is additionally illustrated in FIG. 14A, where the clinical evaluation system 1400 is of a modular design in which disease-specific add-on modules 1402 (e.g., to assess for elevated LVEDP or mPAP, CAD, PH/PAH, abnormal LVEF, HFpEF, and others described herein) are capable of being integrated alone or in multiple instances with a singular platform (i.e., a base system 1404) to realize system 1400's full operation. The modularity allows the clinical evaluation system 1400 to be designed to leverage the same synchronously acquired biophysical signals and data set and base platform to assess for the presence of several different diseases as such disease-specific algorithms are developed, thereby reducing testing and certification time and cost.

In various embodiments, different versions of the clinical evaluation system 1400 may implement the assessment system 103 (FIG. 1) by having included containing different feature computation modules that can be configured for a given disease state(s), medical condition(s), or indicating condition(s) of interest. In another embodiment, the clinical evaluation system 1400 may include more than one assessment system 103 and may be selectively utilized to generate different scores specific to a classifier 116 of that engine 103. In this way, the modules of FIGS. 1 and 14 in a more general sense may be viewed as one configuration of a modular system in which different and/or multiple engines 103, with different and/or multiple corresponding classifiers 116, may be used depending on the configuration of module desired. As such, any number of embodiments of the modules of FIG. 1, with or without PPG waveform-based specific feature(s), may exist.

In FIG. 14A, System 1400 can analyze one or more biophysical-signal data sets (e.g., 110) using machine-learned disease-specific algorithms to assess for the likelihood of elevated LVEDP, as one example, of pathology or abnormal state. System 1400 includes hardware and software components that are designed to work together in combination to facilitate the analysis and presentation of an estimation score using the algorithm to allow a physician to use that score, e.g., to assess for the presence or non-presence of a disease state, medical condition, or an indication of either.

The base system 1404 can provide a foundation of functions and instructions upon which each add-on module 1402 (which includes the disease-specific algorithm) then interfaces to assess for the pathology or indicating condition. The base system 1404 as shown in the example of FIG. 14A includes a base analytical engine or analyzer 1406, a web-service data transfer API 1408 (shown as “DTAPI” 1408), a report database 1410, a web portal service module 1413, and the data repository 111 (shown as 112a).

Data repository 112a, which can be cloud-based, stores data from the signal capture system 102 (shown as 102b). Biophysical signal capture system 102b, in some embodiments, is a reusable device designed as a single unit with a seven-channel lead set and photoplethysmogram (PPG) sensor securely attached (i.e., not removable). Signal capture system 102b, together with its hardware, firmware, and software, provides a user interface to collect patient-specific metadata entered therein (e.g., name, gender, date of birth, medical record number, height, and weight, etc.) to synchronously acquire the patient’s electrical and hemodynamic signals. The signal capture system 102b may securely transmit the metadata and signal data as a single data package directly to the cloud-based data repository. The data repository 112a, in some embodiments, is a secure cloud-based database configured to accept and store the patient-specific data package and allow for its retrieval by the analytical engines or analyzer 1406 or 1414.

Base analytical engine or analyzer 1406 is a secure cloud-based processing tool that may perform quality assessments of the acquired signals (performed via “SQA” module 1416), the results of which can be communicated to the user at the point of care. The base analytical engine or analyzer 1406 may also perform pre-processing (shown via pre-processing module 1418) of the acquired biophysical signals (e.g., 110 - see FIG. 1). Web portal 1413 is a secure web-based portal designed to provide healthcare providers access to their patient’s reports. An example output of the web portal 1413 is shown by visualization 1436. The report databases (RD) 1412 is a secure database and may securely interface and communicate with other systems, such as a hospital or physician-hosted, remotely hosted, or remote electronic health records systems (e.g., Epic, Cerner, Allscrips, CureMD, Kareo, etc.) so that output score(s) (e.g., 118) and related information may be integrated into and saved with the patient’s general health record. In some embodiments, web portal 1413 is accessed by a call center to provide the output clinical information over a telephone. Database 1412 may be accessed by other systems that can generate a report to be delivered via the mail, courier service, personal delivery, etc.

Add-on module 1402 includes a second part 1414 (also referred to herein as the analytical engine (AE) or analyzer 1414 and shown as “AE add-on module” 1414) that operates with the base analytical engine (AE) or analyzer 1406. Analytical engine (AE) or analyzer 1414 can include the main function loop of a given disease-specific algorithm, e.g., the feature computation module 1420, the classifier model 1424 (shown as “Ensemble” module 1424), and the outlier assessment and rejection module 1424 (shown as “Outlier Detection” module 1424). In certain modular configurations, the analytical engines or analyzers (e.g., 1406 and 1414) may be implemented in a single analytical engine module.

The main function loop can include instructions to (i) validate the executing environment to ensure all required environment variables values are present and (ii) execute an analysis pipeline that analyzes a new signal capture data file comprising the acquired biophysical signals to calculate the patient’s score using the disease-specific algorithm. To execute the analysis pipeline, AE add-on module 1414 can include and execute instructions for the various feature modules 114 and classifier module 116 as described in relation to FIG. 1 to determine an output score (e.g., 118) of the metrics associated with the physiological state of a patient. The analysis pipeline in the AE add-on module 1414 can compute the features or parameters (shown as “Feature Computation” 1420) and identifies whether the computed features are outliers (shown as “Outlier Detection” 1422) by providing an outlier detection return for a signal-level response of outlier vs. non-outlier based on the feature. The outliers may be assessed with respect to the training data set used to establish the classifier (of module 116). AE add-on module 1414 may generate the patient’s output score (e.g., 118) (e.g., via classifier module 1424) using the computed values of the features and classifier models. In the example of an evaluation algorithm for the estimation of elevated LVEDP, the output score (e.g., 118) is an LVEDP score. For the estimation of CAD, the output score (e.g., 118) is a CAD score.

The clinical evaluation system 1400 can manage the data within and across components using the web-service DTAPIs 1408 (also may be referred to as HCPP web services in some embodiments). DTAPIs 1408 may be used to retrieve acquired biophysical data sets from and to store signal quality analysis results to the data repository 112a. DTAPIs 1408 may also be invoked to retrieve and provide the stored biophysical data files to the analytical engines or analyzers (e.g., 1406, 1414), and the results of the analytical engine’s analysis of the patient signals may be transferred using DTAPI 1408 to the report database 1410. DTAPIs 1408 may also be used, upon a request by a healthcare professional, to retrieve a given patient data set to the web portal module 1413, which may present a report to the healthcare practitioner for review and interpretation in a secure web-accessible interface.

Clinical evaluation system 1400 includes one or more feature libraries 1426 that store the PPG waveform-based features 120 and various other features of the feature modules 122. The feature libraries 1426 may be a part of the add-on modules 1402 (as shown in FIG. 14A) or the base system 1404 (not shown) and are accessed, in some embodiments, by the AE add-on module 1414.

Further details of the modularity of modules and various configurations are provided in U.S. Provisional Pat. Application no. 63/235,960, filed Aug. 19, 2021, entitled “Modular Disease Assessment System,” which is hereby incorporated by reference herein in its entirety.

### Example Operation of the Modular Clinical Evaluation System

FIG. 14B shows a schematic diagram of the operation and workflow of the analytical engines or analyzers (e.g., 1406 and 1414) of the clinical evaluation system 1400 of FIG. 14A in accordance with an illustrative embodiment.

Signal quality assessment/rejection (1430). Referring to FIG. 14B, the base analytical engine or analyzer 1406 assesses (1430), via SQA module 1416, the quality of the acquired biophysical-signal data set while the analysis pipeline is executing. The results of the assessment (e.g., pass/fail) are immediately returned to the signal capture system’s user interface for reading by the user. Acquired signal data that meet the signal quality requirements are deemed acceptable (i.e., “pass”) and further processed and subjected to analysis for the presence of metrics associated with the pathology or indicating condition (e.g., elevated LVEDP or mPAP, CAD, PH/PAH, abnormal LVEF, HFpEF) by the AE add-on module 1414. Acquired signals deemed unacceptable are rejected (e.g., “fail”), and a notification is immediately sent to the user to inform the user to immediately obtain additional signals from the patient (see FIG. 2).

The base analytical engine or analyzer 1406 performs two sets of assessments for signal quality, one for the electrical signals and one for the hemodynamic signals. The electrical signal assessment (1430) confirms that the electrical signals are of sufficient length, that there is a lack of high-frequency noise (e.g., above 170 Hz), and that there is no power line noise from the environment. The hemodynamic signal assessment (1430) confirms that the percentage of outliers in the hemodynamic data set is below a pre-defined threshold and that the percentage and maximum duration that the signals of the hemodynamic data set are railed or saturated is below a pre-defined threshold.

Feature Value Computation (1432). The AE add-on module 1414 performs feature extraction and computation to calculate feature output values. In the example of the LVEDP algorithm, the AE add-on module 1414 determines, in some embodiments, a total of 446 feature outputs belonging to 18 different feature families (e.g., generated in modules 120 and 122), including the PPG waveform-based features (e.g., generated in module 120). For the CAD algorithm, an example implementation of the AE add-on module 1214 determines a set of features, including 456 features corresponding to the same 18 feature families.

Additional descriptions of the various features, including those used in the LVEDP algorithm and other features and their feature families, are described in U.S. Provisional Pat. Application No. 63/235,960, filed Aug. 23, 2021, entitled “Method and System to Non-Invasively Assess Elevated Left Ventricular End-Diastolic Pressure”; U.S. Provisional Pat. Application No. 63/236,072, filed Aug. 23, 2021, entitled “Methods and Systems for Engineering Visual Features From Biophysical Signals for Use in Characterizing Physiological Systems”; U.S. Provisional Pat. Application No. 63/235,963, filed Aug. 23, 2021, entitled “Methods and Systems for Engineering Power Spectral Features From Biophysical Signals for Use in Characterizing Physiological Systems”; U.S. Provisional Pat. Application No. 63/235,966, filed Aug. 23, 2021, entitled “Method and System for Engineering Rate-Related Features_From Biophysical Signals for Use in Characterizing Physiological Systems”; U.S. Provisional Pat. Application No. 63/235,968, filed Aug. 23, 2021, entitled “Methods and Systems for Engineering Wavelet-Based Features From Biophysical Signals for Use in Characterizing Physiological Systems”; U.S. Provisional Pat. Application No. 63/130,324, titled “Method and System to Assess Disease Using Cycle Variability Analysis of Cardiac and Photoplethysmographic Signals”; U.S. Provisional Pat. Application No. 63/236,193, filed Aug. 23, 2021, entitled “Methods and Systems for Engineering Cardiac Waveform Features From Biophysical Signals for Use in Characterizing Physiological Systems”; U.S. Provisional Pat. Application No. 63/235,974, filed Aug. 23, 2021, entitled “Methods and Systems for Engineering Conduction Deviation Features From Biophysical Signals for Use in Characterizing Physiological Systems,” each of which is hereby incorporated by reference herein in its entirety.

Classifier Output Computation (1434). The AE add-on module 1414 then uses the calculated feature outputs in classifier models (e.g., machine-learned classifier models) to generate a set of model scores. The AE add-on module 1414 joins the set of model scores in an ensemble of the constituent models, which, in some embodiments, averages the output of the classifier models as shown in Equation 4 in the example of the LVEDP algorithm.

$$E n s e m b l e e s t i m a t i o n = \frac{M o d e {l}_{1} + M o d e {l}_{2} + … + M o d e {l}_{n}}{n}$$

In some embodiments, classifier models may include models that are developed based on ML techniques described in U.S. Patent Publication No. 20190026430, entitled “Discovering Novel Features to Use in Machine Learning Techniques, such as Machine Learning Techniques for Diagnosing Medical Conditions”; or U.S. Patent Publication No. 20190026431, entitled “Discovering Genomes to Use in Machine Learning Techniques,” each of which is hereby incorporated by reference herein in its entirety.

In the example of the LVEDP algorithm, thirteen (13) machine-learned classifier models are each calculated using the calculated feature outputs. The 13 classifier models include four ElasticNet machine-learned classifier models, four RandomForestClassifier machine-learned classifier models, and five extreme gradient boosting (XGB) classifier models. In some embodiments, the patient’s metadata information, such as age, gender, and BMI value, may be used. The output of the ensemble estimation may be a continuous score. The score may be shifted to a threshold value of zero by subtracting the threshold value for presentation within the web portal. The threshold value may be selected as a trade-off between sensitivity and specificity. The threshold may be defined within the algorithm and used as the determination point for test positive (e.g., “Likely Elevated LVEDP”) and test negative (e.g., “Not Likely Elevated LVEDP”) conditions.

In some embodiments, the analytical engine or analyzer can fuse the set of model scores with a body mass index-based adjustment or an adjustment based on age or gender. For example, the analytical engine or analyzer can average the model estimation with a sigmoid function of the patient BMI having the form sigmoid(x) =

$$\frac{1}{1 + {e}^{- x}} .$$

Physician Portal Visualization (1436). The patient’s report may include a visualization 1436 of the acquired patient data and signals and the results of the disease analyses. The analyses are presented, in some embodiments, in multiple views in the report. In the example shown in FIG. 14B, the visualization 1436 includes a score summary section 1440 (shown as “Patient LVEDP Score Summary” section 1440), a threshold section 1442 (shown as “LVEDP Threshold Statistics” section 1442), and a frequency distribution section 1444 (shown as “Frequency Distribution” section 1408). A healthcare provider, e.g., a physician, can review the report and interpret it to provide a diagnosis of the disease or to generate a treatment plan.

The healthcare portal may list a report for a patient if a given patient’s acquired signal data set meets the signal quality standard. The report may indicate a disease-specific result (e.g., elevated LVEDP) being available if the signal analysis could be performed. The patient’s estimated score (shown via visual elements 118a, 118b, 118c) for the disease-specific analysis may be interpreted relative to an established threshold.

In the score summary section 1440 shown in the example of FIG. 14B, the patient’s score 118a and associated threshold are superimposed on a two-tone color bar (e.g., shown in section 1440) with the threshold located at the center of the bar with a defined value of “0” representing the delineation between test positive and test negative. The left of the threshold may be lightly shaded light and indicates a negative test result (e.g., “Not Likely Elevated LVEDP”), while to the right of the threshold may be darkly shaded to indicate a positive test result (e.g., “Likely Elevated LVEDP”).

The threshold section 1442 shows reported statistics of the threshold as provided to a validation population that defines the sensitivity and specificity for the estimation of the patient score (e.g., 118). The threshold is the same for every test regardless of the individual patient’s score (e.g., 118), meaning that every score, positive or negative, may be interpreted for accuracy in view of the provided sensitivity and specificity information. The score may change for a given disease-specific analysis as well with the updating of the clinical evaluation.

The frequency distribution section 1444 illustrates the distribution of all patients in two validation populations (e.g., (i) a non-elevated population to indicate the likelihood of a false positive estimation and (ii) an elevated population to indicate a likelihood of a false negative estimation). The graphs (1446, 1448) are presented as smooth histograms to provide context for interpreting the patient’s score 118 (e.g., 118b, 118c) relative to the test performance validation population patients.

The frequency distribution section 1440 includes a first graph 1446 (shown as “Non-Elevated LVEDP Population” 1446) that shows the score (118b), indicating the likelihood of the non-presence of the disease, condition, or indication, within a distribution of a validation population having non-presence of that disease, condition, or indication and a second graph 1448 (shown as “Elevated LVEDP Population” 1448) that shows the score (118c), indicates the likelihood of the presence of the disease, condition, or indication, within a distribution of validation population having the presence of that disease, condition, or indication. In the example of the assessment of elevated LVDEP, the first graph 1446 shows a non-elevated LVEDP distribution of the validation population that identifies the true negative (TN) and false positive (FP) areas. The second graph 1448 shows an elevated LVEDP distribution of the validation population that identifies the false negative (TN) and true positive (FP) areas.

The frequency distribution section 1440 also includes interpretative text of the patient’s score relative to other patients in a validation population group (as a percentage). In this example, the patient has an LVEDP score of -0.08, which is located to the left side of the LVEDP threshold, indicating that the patient has “Not Likely Elevated LVEDP.”

The report may be presented in the healthcare portal, e.g., to be used by a physician or healthcare provider in their diagnosis for indications of left-heart failure. The indications include, in some embodiments, a probability or a severity score for the presence of a disease, medical condition, or an indication of either.

Outlier Assessment and Rejection Detection (1438). Following the AE add-on module 1414 computing the feature value outputs (in process 1432) and prior to their application to the classifier models (in process 1434), the AE add-on module 1414 is configured in some embodiments to perform outlier analysis (shown in process 1438) of the feature value outputs. Outlier analysis evaluation process 1438 executes a machine-learned outlier detection module (ODM), in some embodiments, to identify and exclude anomalous acquired biophysical signals by identifying and excluding anomalous feature output values in reference to the feature values generated from the validation and training data. The outlier detection module assesses for outliers that present themselves within sparse clusters at isolated regions that are out of distribution from the rest of the observations. Process 1438 can reduce the risk that outlier signals are inappropriately applied to the classifier models and produce inaccurate evaluations to be viewed by the patient or healthcare provider. The accuracy of the outlier module has been verified using hold-out validation sets in which the ODM is able to identify all the labeled outliers in a test set with the acceptable outlier detection rate (ODR) generalization.

While the methods and systems have been described in connection with certain embodiments and specific examples, it is not intended that the scope be limited to the particular embodiments set forth, as the embodiments herein are intended in all respects to be illustrative rather than restrictive. The PPG waveform-based features discussed herein may ultimately be employed to make, or to assist a physician or other healthcare provider in making, noninvasive diagnoses or determinations of the presence or non-presence and/or severity of other diseases, medical conditions, or indication of either, such as, e.g., coronary artery disease, pulmonary hypertension and other pathologies as described herein using similar or other development approaches. In addition, the example analysis, including the PPG waveform-based features, can be used in the diagnosis and treatment of other cardiac-related pathologies and indicating conditions as well as neurological-related pathologies and indicating conditions, such assessment can be applied to the diagnosis and treatment (including, surgical, minimally invasive, and/or pharmacologic treatment) of any pathologies or indicating conditions in which a biophysical signal is involved in any relevant system of a living body. One example in the cardiac context is the diagnosis of CAD, and other diseases, medical conditions, or indicating conditions disclosed herein and its treatment by any number of therapies, alone or in combination, such as the placement of a stent in a coronary artery, the performance of an atherectomy, angioplasty, prescription of drug therapy, and/or the prescription of exercise, nutritional and other lifestyle changes, etc. Other cardiac-related pathologies or indicating conditions that may be diagnosed include, e.g., arrhythmia, congestive heart failure, valve failure, pulmonary hypertension (e.g., pulmonary arterial hypertension, pulmonary hypertension due to left heart disease, pulmonary hypertension due to lung disease, pulmonary hypertension due to chronic blood clots, and pulmonary hypertension due to other diseases such as blood or other disorders), as well as other cardiac-related pathologies, indicating conditions and/or diseases. Non-limiting examples of neurological-related diseases, pathologies or indicating conditions that may be diagnosed include, e.g., epilepsy, schizophrenia, Parkinson’s Disease, Alzheimer’s Disease (and all other forms of dementia), autism spectrum (including Asperger syndrome), attention deficit hyperactivity disorder, Huntington’s Disease, muscular dystrophy, depression, bipolar disorder, brain/spinal cord tumors (malignant and benign), movement disorders, cognitive impairment, speech impairment, various psychoses, brain/spinal cord/nerve injury, chronic traumatic encephalopathy, cluster headaches, migraine headaches, neuropathy (in its various forms, including peripheral neuropathy), phantom limb/pain, chronic fatigue syndrome, acute and/or chronic pain (including back pain, failed back surgery syndrome, etc.), dyskinesia, anxiety disorders, indicating conditions caused by infections or foreign agents (e.g., Lyme disease, encephalitis, rabies), narcolepsy and other sleep disorders, post-traumatic stress disorder, neurological conditions/effects related to stroke, aneurysms, hemorrhagic injury, etc., tinnitus and other hearing-related diseases/indicating conditions and vision-related diseases/indicating conditions.

In addition, the clinical evaluation system described herein may be configured to analyze biophysical signals such as an electrocardiogram (ECG), electroencephalogram (EEG), gamma synchrony, respiratory function signals, pulse oximetry signals, perfusion data signals; quasi-periodic biological signals, fetal ECG signals, blood pressure signals; cardiac magnetic field signals, heart rate signals, among others.

Further examples of processing that may be used with the exemplified method and system disclosed herein are described in: U.S. Pat. Nos. 9,289,150; 9,655,536; 9,968,275; 8,923,958; 9,408,543; 9,955,883; 9,737,229; 10,039,468; 9,597,021; 9,968,265; 9,910,964; 10,672,518; 10,566,091; 10,566,092; 10,542,897; 10,362,950; 10,292,596; 10,806,349; U.S. Pat. Publication Nos. 2020/0335217; 2020/0229724; 2019/0214137; 2018/0249960; 2019/0200893; 2019/0384757; 2020/0211713; 2019/0365265; 2020/0205739; 2020/0205745; 2019/0026430; 2019/0026431; PCT Publication Nos. WO2017/033164; WO2017/221221; WO2019/130272; WO2018/158749; WO2019/077414; WO2019/130273; WO2019/244043; WO2020/136569; WO2019/234587; WO2020/136570; WO2020/136571; U.S. Pat. Application Nos. 16/831,264; 16/831,380; 17/132869; PCT Application Nos. PCT/IB2020/052889; PCT/IB2020/052890, each of which is hereby incorporated by reference herein in its entirety.

The following patents, applications, and publications as listed below and throughout this document are hereby incorporated by reference in their entirety herein.

### LIST OF REFERENCES

J. M. Cho, Y. K. Sung, K. W. Shin, D. J. Jung, Y. S. Kim and N. H. Kim, “A preliminary study on photoplethysmogram (PPG) signal analysis for reduction of motion artifact in frequency domain,” 2012 IEEE-EMBS Conference on Biomedical Engineering and Sciences, Langkawi, 2012, pp. 28-33, doi: 10.1109/IECBES.2012.6498141.

What is claimed is:

1. A method to non-invasively assess a disease state, abnormal condition, or an indication of either of a subject, the method comprising:
obtaining, by one or more processors, a biophysical signal data set of the subject, the biophysical signal data set comprising one or more photoplethysmographic signals;
determining, by the one or more processors, values of one or more waveform associated properties of the one or more photoplethysmographic signals; and
determining, by the one or more processors, an estimated value for a presence of the disease state, abnormal condition, or an indication of either based, in part, on the determined values of the one or more waveform associated properties, wherein the estimated value for the of the disease state, abnormal condition, or indication of either is used in a model to non-invasively estimate a presence of an expected disease state, abnormal condition, or indication of either,
wherein the estimated value is subsequently outputted for use in a diagnosis of the expected disease state or condition or to direct treatment of the expected disease state, abnormal condition, or indication of either.

2. The method of claim 1, wherein the step of determining the values of one or more waveform associated properties comprises:
determining, by the one or more processors, one or more values of amplitude-associated features extracted from a photoplethysmographic signal or a derivative thereof, wherein the one or more amplitude-associated features include a feature selected from the group consisting of:
a feature comprising a statistical assessment of fiduciary landmarks determined in at least one of the one or more photoplethysmographic signals;
a feature comprising a statistical assessment of fiduciary landmarks determined in a velocityplethysmographic signal derived from at least one of the one or more photoplethysmographic signal; and
a feature comprising a statistical assessment of fiduciary landmarks determined in an acceleration-plethysmographic signal derived from at least one of the one or more photoplethysmographic signal.

3. The method of claim 2, wherein the fiduciary landmarks determined in the at least one of the one or more photoplethysmographic signals comprise pulse base landmarks, diastolic peak landmarks, systolic peak landmarks, minimum landmarks, minimums proximal to peaks landmarks.

4. The method of claim 2, wherein the fiduciary landmarks determined in the velocityplethysmographic signal or the accelerationplethysmographic signal comprise pulse base landmarks, peak landmarks, or minimum landmarks.

5. The method of claim 2, wherein the statistical assessment is selected from the group consisting of a mean of the amplitude of the respective signal, a standard deviation of the amplitude of the respective signal, a maximum amplitude of the respective signal, a minimum amplitude of the respective signal, and a minimum amplitude of an assessed peak in the respective signal.

6. The method of claim 5, wherein the step of determining the values of the one or more waveform associated properties comprises:
determining, by the one or more processors, one or more values of duration-associated features extracted from a photoplethysmographic signal or a derivative thereof, wherein the one or more amplitude-associated features include a feature selected from the group consisting of:
a feature comprising a statistical assessment of a beat-to-beat duration of fiduciary landmarks determined in at least one of the one or more photoplethysmographic signals;
a feature comprising a statistical assessment of the beat-to-beat duration of fiduciary landmarks determined in the velocityplethysmographic signal derived from at least one of the one or more photoplethysmographic signals; and
a feature comprising a statistical assessment of the beat-to-beat duration of fiduciary landmarks determined in the acceleration-plethysmographic signal derived from at least one of the one or more photoplethysmographic signals.

7. The method of claim 1, wherein the step of determining the values of one or more waveform associated properties comprises:
determining, by the one or more processors, one or more values of duration-associated features extracted from a photoplethysmographic signal or a derivative thereof, wherein the one or more amplitude-associated features include a feature selected from the group consisting of:
a feature comprising a statistical assessment of duration between fiduciary landmarks in periodic beats determined in at least one of the one or more photoplethysmographic signals;
a feature comprising a statistical assessment of duration between fiduciary landmarks in periodic beats determined in a velocityplethysmographic signal derived from at least one of the one or more photoplethysmographic signals; and
a feature comprising a statistical assessment of duration between fiduciary landmarks in periodic beats determined in the accelerationplethysmographic signal derived from at least one of the one or more photoplethysmographic signals.

8. The method of claim 1, wherein the step of determining the values of one or more waveform associated properties comprises:
determining, by the one or more processors, one or more values of waveform geometry-associated features extracted from a photoplethysmographic signal, wherein the one or more waveform geometry-associated features comprise a statistical assessment of a waveform-geometric assessment of one or more triangles defined among fiduciary landmarks determined in at least one of the one or more photoplethysmographic signals.

9. The method of claim 8, wherein the one or more triangles are selected from the group consisting of:
a triangle defined between the pulse base landmarks, the systolic peak landmarks, and the diastolic peak landmarks determined in the at least one of the one or more photoplethysmographic signals.

10. The method of claim 9, wherein the step of determining the values of one or more waveform associated properties comprises:
determining, by the one or more processors, one or more values of SpO₂-associated features extracted from a photoplethysmographic signal, wherein the one or more SpO₂-associated features comprise a statistical assessment of a vector defined as a ratio of AC and DC components determined in at least two of the photoplethysmographic signals.

11. The method of claim 1 further comprising:
causing, by the one or more processors, generation of a visualization of the estimated value for the presence of the disease state, abnormal condition, or the indication of either, wherein the generated visualization is rendered and displayed at a display of a computing device and/or presented in a report.

12. The method of claim 1, wherein the values of one or more waveform associated properties are used in the model selected from the group consisting of a linear model, a decision tree model, a random forest model, a support vector machine model, and a neural network model.

13. The method of claim 12, wherein the model further includes features selected from the group consisting of:
one or more depolarization or repolarization wave propagation associated features;
one or more depolarization wave propagation deviation associated features;
one or more cycle variability associated features;
one or more dynamical system associated features;
one or more cardiac waveform topologic and variations associated features;
one or more PPG waveform topologic and variations associated features;
one or more cardiac or PPG signal power spectral density associated features;
one or more cardiac or PPG signal visual associated features; and
one or more predictability features.

14. The method of claim 1, wherein the disease state or abnormal condition is selected from the group consisting of coronary artery disease, pulmonary hypertension, pulmonary arterial hypertension, pulmonary hypertension due to left heart disease, rare disorders that lead to pulmonary hypertension, left ventricular heart failure or left-sided heart failure, right ventricular heart failure or right-sided heart failure, systolic heart failure, diastolic heart failure, ischemic heart disease, and arrhythmia.

15. The method of claim 1 further comprising:
acquiring, by one or more acquisition circuits of a measurement system, voltage gradient signals over the one or more channels, wherein the voltage gradient signals are acquired at a frequency greater than about 1 kHz; and
generating, by the one or more acquisition circuits, the obtained biophysical data set from the acquired voltage gradient signals.

16. The method of claim 1 further comprising:
acquiring, by one or more acquisition circuits of a measurement system, one or more photoplethysmographic signals; and
generating, by the one or more acquisition circuits, the obtained biophysical data set from the acquired voltage gradient signals.

17. The method of claim 1, wherein the one or more processors are located in a cloud platform.

18. The method of claim 1, wherein the one or more processors are located in a local computing device.

19. A system comprising:
a processor; and
a memory having instructions stored thereon, wherein execution of the instructions by the processor causes the processor to:
obtain a biophysical signal data set of a subject, a biophysical signal data set comprising one or more photoplethysmographic signals;
determine values of one or more waveform associated properties of the one or more photoplethysmographic signals; and
determine an estimated value for a presence of the disease state, abnormal condition, or an indication of either based, in part, on the determined values of the one or more waveform associated properties, wherein the estimated value for the of the disease state, abnormal condition, or indication of either is used in a model to non-invasively estimate a presence of an expected disease state, abnormal condition, or indication of either,
wherein the estimated value is subsequently outputted for use in a diagnosis of the expected disease state or condition or to direct treatment of the expected disease state, abnormal condition, or indication of either.

20. A non-transitory computer-readable medium having instructions stored thereon, wherein execution of the instructions by a processor causes the processor to:
obtain a biophysical signal data set of a subject, a biophysical signal data set comprising one or more photoplethysmographic signals;
determine values of one or more waveform associated properties of the one or more photoplethysmographic signals; and
determine an estimated value for a presence of the disease state, abnormal condition, or an indication of either based, in part, on the determined values of the one or more waveform associated properties, wherein the estimated value for the of the disease state, abnormal condition, or indication of either is used in a model to non-invasively estimate a presence of an expected disease state, abnormal condition, or indication of either,
wherein the estimated value is subsequently outputted for use in a diagnosis of the expected disease state or condition or to direct treatment of the expected disease state, abnormal condition, or indication of either.
